(19) United States
(12) Patent Application Publication
(10) Pub. No.: US 2026/0123870 A1
(43) Pub. Date: May 7, 2026

(54) Mesoscopic Electrophysiology Device and Related Methods

(71) Applicant: University of Pittsburgh - Of the Commonwealth System of Higher Education, Pittsburgh, PA (US)

(72) Inventor: Tobias Teichert, Pittsburgh, PA (US)

(21) Appl. No.: 19/381,421

(22) Filed: Nov. 6, 2025

Related U.S. Application Data
(60) Provisional application No. 63/717,487, filed on Nov. 7, 2024.

(51) Int. Cl. A61B 5/291; A61B 5/00; A61B 5/251; A61B 5/262; A61N 1/05

(52) U.S. Cl. CPC A61B 5/291; A61B 5/251; A61B 5/262; A61B 5/4848; A61B 5/6814; A61B 5/6847; A61B 5/7235; A61N 1/0529; A61B 2560/0468; A61B 2560/06; A61B 2562/046

(57) ABSTRACT

Provided herein is a mesoscopic electrophysiological system including a structural system sized and configured to be attached to a head of a patient and to hold a plurality of intracranial electrode shafts in predetermined positions around the head of the patient, a plurality of mesoscopic intracranial electrode shafts that includes a first plurality of electrode contacts configured to measure local field potentials (LFPs) at a plurality of locations in a brain of a patient, wherein each electrode shaft of the plurality of intracranial electrode shafts includes a second plurality of electrode contacts, and a plurality of guide pins sized and configured to receive the plurality of mesoscopic intracranial electrode shafts positioned therein.

### CROSS REFERENCE TO RELATED APPLICATION

This application claims the benefit of U.S. Provisional Ser. No. 63/717,487, filed Nov. 7, 2024, the content of which is incorporated herein by reference in its entirety.

### STATEMENT REGARDING FEDERALLY SPONSORED RESEARCH

This invention was made with government support under MH114223 awarded by the National Institutes of Health. The government has certain rights in the invention.

### BACKGROUND

### Field

Provided herein are devices, systems, and methods for monitoring brain activity and effects of interventions on brain activity.

### Description of Related Art

The brain is a highly interconnected organ whose functional connectome can only be understood by recording from the entire brain in parallel, rather than parts of it in sequence. Hence, the ability to record full-brain functional connectivity maps with sub-millimeter spatial resolution is at the core of the appeal of fMRI. However, the temporal resolution of the fMRI functional connectome is limited by the relatively slow hemodynamic response. In contrast to functional magnetic resonance imaging (fMRI), electrophysiological approaches have excellent temporal resolution. However, they either 1) do not cover the entire brain, as is the case for microscopic intracranial recordings, or 2) have relatively poor spatial resolution, as is the case for macroscopic recordings, such as electroencephalogram (EEG). Here, we aimed to develop a new mesoscopic electrophysiological recording platform that combines the excellent temporal resolution of electrophysiology with the whole-brain coverage of fMRI, while maintaining sufficient spatial resolution for network-based connectivity analyses. To date, such mesoscopic whole-brain approaches have been largely neglected in the non-human primate, thus leaving a valuable source of information untapped.

### SUMMARY

Accordingly, it is an object of the presently disclosed subject matter to provide a mesoscopic electrophysiological system for monitoring brain activity and effects of interventions on brain activity.

Further non-limiting embodiments or aspects will be set forth in the following numbered clauses:
Clause 1: A mesoscopic electrophysiological system for recording electrophysiological aspects of a brain of a patient, comprising: a structural system sized and configured to be attached to a head of a patient and to hold a plurality of intracranial electrode shafts in predetermined positions around the head of the patient; a plurality of mesoscopic intracranial electrode shafts that comprises a first plurality of electrode contacts configured to measure local field potentials (LFPs) at a plurality of locations in a brain of a patient, wherein each electrode shaft of the plurality of intracranial electrode shafts comprises a second plurality of electrode contacts; and a plurality of guide pins sized and configured to receive the plurality of mesoscopic intracranial electrode shafts positioned therein; wherein the first plurality of electrode contacts forms a three-dimensional grid within the head of the patient.
Clause 2: The mesoscopic electrophysiological system of clause 1, wherein the second plurality of contacts is configured to provide electrical micro-stimulation to the plurality of locations in the brain of the patient.
Clause 3: The mesoscopic electrophysiological system of clauses 1 or 2,wherein each guide pin of the plurality of guide pins comprises an interior channel, and wherein a sterile grease is provided in the interior channel of one or more guide pins of the plurality of guide pins.
Clause 4: The mesoscopic electrophysiological system of any of clauses 1-3, wherein each guide pin of the plurality of guide pins comprises a vent connected to the interior channel, and wherein the vent provides a path for the sterile grease to travel when a mesoscopic intracranial electrode shaft is positioned within the interior channel of a guide pin.
Clause 5: The mesoscopic electrophysiological system of any of clauses 1-4, wherein the second plurality of electrode contacts have variable inter-electrode spacing in range between 250 μm and 3.5 mm.
Clause 6: The mesoscopic electrophysiological system of any of clauses 1-5, further comprising: at least one processor configured to: receive data associated with the LFPs in the brain of the patient from a subset of the first plurality of electrode contacts; generate a representation of a functional connectivity profile for the patient based on the data associated with the LFPs in the brain of the patient; and transmit the representation of the functional connectivity profile for the patient to a communications device.
Clause 7: The mesoscopic electrophysiological system of any of clauses 1-6, wherein the first plurality of electrode contacts is configured to provide at least 1,000 channels for measuring the LFPs at the plurality of locations in the brain of the patient.
Clause 8: The mesoscopic electrophysiological system of any of clauses 1-7, wherein the first plurality of electrode contacts is configured to measure the LFPs at the plurality of locations in the brain of the patient at a sampling rate in a range between 20 kHz and 30 kHz.
Clause 9: The mesoscopic electrophysiological system of any of clauses 1-8, wherein the structural system comprises: a crown component comprising a top section and an annular section, wherein the crown component is configured to be anchored to a skull of the patient via fasteners inserted through the annular section; a semi-spherical grid component comprising a first structure having a first plurality of apertures, wherein the structure corresponds to a shape of a skull of the patient, and wherein the semi-spherical grid component is positioned within the crown component; a wall component comprising a base section and a top section, wherein the base section is configured to connect to the top section of the crown component; and a grid component comprising a second structure having a second plurality of apertures, wherein the grid component is configured to fit within the wall component and is configured to receive the plurality of guide pins within the second plurality of apertures, and wherein the second plurality of apertures align with at least a portion of the first plurality of apertures to allow for the plurality of intracranial electrode shafts to be inserted into the brain of the patient.
Clause 10: The mesoscopic electrophysiological system of any of clauses 1-9, wherein the second plurality of apertures of the grid component are spaced apart in an antero-posterior direction and medio-later direction by a dimension in a range between 4 mm and 5 mm.
Clause 11: The mesoscopic electrophysiological system of any of clauses 1-10, wherein one or more apertures of the second plurality of apertures has an inner diameter equal to 1.5 mm.
Clause 12: The mesoscopic electrophysiological system of any of clauses 1-11,wherein the structural system comprises: a cap component comprising a cap section and a cap top section, wherein the cap section comprises a wall having a peripheral aperture, wherein the wall defines a main aperture, wherein the cap section is configured to connect to the top section of the wall component, wherein the cap top section is configured to cover the main aperture of the wall, and wherein the peripheral aperture is sized and configured to allow for cables that connect to the intracranial electrode shafts to pass therethrough.
Clause 13: The mesoscopic electrophysiological system of any of clauses 1-12, wherein the semi-spherical grid component comprises a plurality of electroencephalogram (EEG) electrodes that are positioned to contact a surface of the head of the patient when the structural system is placed on the head of the patient.
Clause 14: A mesoscopic electrophysiological system for recording electrophysiological aspects of a brain of a patient, comprising: a structural system sized and configured to be attached to a head of a patient and to hold a plurality of intracranial electrode shafts in predetermined positions around the head of the patient; a plurality of mesoscopic intracranial electrode shafts that comprises a plurality of electrode contacts configured to measure local field potentials (LFPs) at a plurality of locations in a brain of a patient; and a plurality of guide pins sized and configured to receive the plurality of mesoscopic intracranial electrode shafts positioned therein; wherein the plurality of electrode contacts forms a three-dimensional grid within the head of the patient.
Clause 15: The mesoscopic electrophysiological system of clause 14, wherein each guide pin of the plurality of guide pins comprises an interior channel, and wherein a sterile grease is provided in the interior channel of one or more guide pins of the plurality of guide pins.
Clause 16: The mesoscopic electrophysiological system of clauses 14 or 15, wherein each guide pin of the plurality of guide pins comprises a vent connected to the interior channel, and wherein the vent provides a path for the sterile grease to travel when a mesoscopic intracranial electrode shaft is positioned within the interior channel of a guide pin.
Clause 17: The mesoscopic electrophysiological system of any of clauses 14-16, wherein the second plurality of electrode contacts have variable inter-electrode spacing in range between 250 μm and 3.5 mm.
Clause 18: The mesoscopic electrophysiological system of any of clauses 14-17, wherein the first plurality of electrode contacts is configured to provide at least 1,000 channels for measuring the LFPs at the plurality of locations in the brain of the patient, and wherein the plurality of electrode contacts is configured to measure the LFPs at the plurality of locations in the brain of the patient at a sampling rate in a range between 20 kHz and 30 kHz.
Clause 19: A method of monitoring a plurality of local field potentials (LFPs) within a patient's brain, comprising: affixing, to the patient, a mesoscopic electrophysiological system for recording electrophysiological aspects of a brain of a patient, the system comprising: a structural system sized and configured to be attached to a head of a patient and to hold a plurality of intracranial electrode shafts in predetermined positions around the head of the patient; a plurality of mesoscopic intracranial electrode shafts that comprises a plurality of electrode contacts configured to measure the LFPs at a plurality of locations in a brain of a patient, the plurality of electrode contacts forming a three-dimensional grid within the head of the patient; a plurality of guide pins sized and configured to receive the plurality of mesoscopic intracranial electrode shafts positioned therein; at least one processor configured to: receive data associated with the LFPs in the brain of the patient from a subset of the first plurality of electrode contacts; generate a representation of a functional connectivity profile for the patient based on the data associated with the LFPs in the brain of the patient; and transmit the representation of the functional connectivity profile for the patient to a communications device; and detecting, with the plurality of electrode contacts, a plurality of baseline LFPs.
Clause 20: The method of clause 19, further comprising, after detecting the plurality of baseline LFPs: administering to the patient a compound of interest and detecting, with the first plurality of electrode contacts, a plurality of post-administration LFPs.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1C show A: an exploded design of the MePhys platform with the main structural and functional elements: crown, spider, wall, grid, baseplate, headstage, and cap. B: a schematic of the electrode-guidepin-grid system. C: the intracranial contacts (small blue dots) of the MePhys prototype are arranged in 14 coronal slices from posterior to anterior from bottom right to top left. The antero-posterior position of the slices is indicated by horizontal lines in the top-down schematic of the monkey brain. Panels for slices no. 1 and no. 2 were omitted because they do not contain any currently functional electrode contacts. The electroencephalogram (EEG) electrodes (large blue dots) are depicted in the top-down schematic at the bottom right. This layout is to be used in subsequent figures to display the hemisphere-wide results by changing the size and color of the dots in accordance with the data for each channel.

FIG. 1D shows an implementation of a procedure for applying a plurality of guide pins (e.g., guide pins 114) to a brain of a patient using a grid component of a mesoscopic electrophysiological system.

FIG. 2A-2D show spatial distribution of auditory-evoked electric fields. A: hemisphere-wide auditory-evoked electric fields. The top features a butterfly plot with time-resolved activity of all channels; the red, orange, and blue lines indicate the time-points displayed in the three panels below. The mapping of color to potential is indicated on the y-axis of the butterfly plot. In addition, the size of the dots is scaled by the absolute value of the potential at each contact to further highlight channels with strong responses. The first activation of auditory cortex occurs ˜9 ms after sound onset (slice no. 7). Activity in motor and prefrontal cortex reaches its first surface-positive peak 17 ms after sound onset (slice nos. 8 and 9). A second peak with inverted polarity emerges around 38 ms after sound onset. Note the dipole-like structure of the fields in auditory and motor cortex. For visualization purposes, the evoked potentials at the EEG electrodes were multiplied by a factor of 2. B: a map of the strength of the evoked fields measured as the standard deviation of the time resolved evoked responses over the first 250 ms of the response. The inset depicts the distribution of evoked field strength across all electrodes and defines the relationship between color and field strength (0 μV: gray, 200 μV: red). C: the attenuation of evoked responses after administration of a subanesthetic dose of ketamine. Note that responses in auditory cortex are not attenuated. The inset again depicts the distribution of values across all channels. D: the attenuation of responses after a subanesthetic dose of midazolam is much more pronounced (note different color scale) and strongly attenuates responses in auditory cortex.

FIG. 3A-3D show movement-related fields and β-band desynchronization. A: a schematic of the delayed tone-discrimination task. B: wavelet decomposition of F1 and CB activity around the time of lever release, i.e., response onset, shows an enhancement of a power and a subsequent reduction of b power. C: timing of β-band response-related desynchronization in seven example regions (CB, cerebellum; S1, primary somatosensory Cx; Cd, caudate; F1, primary motor Cx; F5, ventral premotor Cx; A1, primary auditory Cx; R, rostral auditory Cx). The earliest drop in b power (defined as a reduction to less than half the power of control trials without a response; dotted line at y=−1) is observed in F1, ˜190 ms after tone onset/120 ms prior to response onset. Cerebellum and F5 had latencies that were ˜25 ms longer than F1. Somatosensory cortex reached criterion ˜50 ms before response onset. Spatial maps of response-related β-band desynchronization (D-E) and response-related evoked fields at 52 ms prior to response onset.

FIG. 4A-4H show distribution of dominant frequencies in the monkey brain. A-C and F-H: example traces and amplitude spectra for 10 channels during control (black) and after injection of ketamine (red). D-E: for each channel, dominant frequency was defined as the frequency with the strongest power. Color-coded dominant frequency (blue [˜3 Hz]—green [˜10 Hz]—yellow [˜15 Hz]—red [˜25 Hz]) across the hemisphere is depicted during control (left) and after ketamine (right). The insets depict the density distribution of dominant frequency values across all channels. The color of the dots defines the color map (e.g., blue for dominant frequencies between 1 and 3 Hz). During control, the mode of the distribution of dominant frequencies is at ˜3.5 Hz (blue vertical line). This peak is carried mostly by channels in occipital and parietal cortex. Channels in motor cortex and cerebellum are dominated by low b (light green/yellow/orange). Channels in and around orbitofrontal cortex are dominated by low a (green). Overall, ketamine shifted the distribution of dominant frequencies upwards (cyan vertical bar at ˜6.5 Hz). Note the emergence of channels around motor cortex with a dominant frequency around 25 Hz. In most cases, the effect of ketamine was to blunt exiting peaks of power. In rare instances, we see the emergence of new peaks (e.g., somatosensory cortex or LGN).

FIG. 5A-5G show Intrinsic coupling. A-B: each panel shows the z-scored temporal correlation of one seed channel (red arrow) with all other channels. Ai: the cerebellum seed confirms the existence of a functional link between cerebellum and motor cortex. The rapid transition between channels with strong positive and negative correlations in and around motor cortex reflects their location either above or below a dipole in motor cortex. Aii: the seed in auditory cortex identified functional connections along the entire superior temporal plane, extending all the way to the temporal pole and with motor cortex and ventrolateral prefrontal cortex. Biii: the amygdala seed revealed the expected functional connections to hippocampal areas and the ventral visual path, and the ventral striatum. Biv: the seed in area 7a revealed a more complicated map with functional connections in hippocampal regions, dorsal prefrontal cortex, and anterior insula. Ci: the matrix of temporal correlations of the raw data for all pairs of channels. Intracranial channels are grouped either by coronal or sagittal slices (above and below the main diagonal, respectively). Within each slice, channels are sorted from medial to lateral or posterior to anterior, respectively. Bands of positive correlation along the main and secondary diagonals indicate stronger correlation for channels that are close to each other. Dii: correlation plotted as a function of distance between each pair of electrodes. Diii: normalized eigenvalues and cumulative percent variance explained by successive principal components. Dimensionality was defined as the number of components needed to explain 80% of the variance (gray lines). E-G: dimensionality (y-axis) of functional connectomes separated by carrier frequencies and mode before and after injection of ketamine (left), midazolam (middle), and falling asleep (right).

FIG. 6A-6E show that the β-band motor network is decoupled by ketamine. A: amplitude spectra of cerebellum and motor cortex before and after injection of ketamine. B: example snippets of β band activity (black) and β envelope (blue). C: β-band cross-correlations before (black) and after (red) injection of ketamine. D: map of channels in eight motor and two control regions (CB, cerebellum; PC, parietal cx; Cd, caudate; GP, globus pallidus; Put, putamen; Cl, claustrum; F1, primary motor cortex; F2, secondary motor cx; F5, suppl. motor cx; PF, prefrontal cx.). E: cross-correlation strength (dot size) and lag (dot color) for all pairs of regions before (top) and after (bottom) injection of ketamine (left), injection of midazolam (middle), or falling asleep (right).

FIG. 7A-7B show that ketamine disrupts the emergence of stable recurrent brain-wide states. Spatial correlation matrices during rest computed from all four lowpass envelopes simultaneously before and after ketamine (7A left panel and 7B left panel) and before and after midazolam (7A right panel and 7B right panel). Color indicates the similarity of the spatial maps at different time points in the recording session. The thickening of the main diagonal into square-like patches along the main diagonal indicate that between periods of change, the brain settles into relatively stable periods that can last tens of seconds. The patches with strong positive correlations off of the main diagonal indicate that the same stable states occur at different points in time. Their square-like shape indicates relatively brisk transitions between states. The emergence of the stable states is largely abolished by ketamine (7B left panel), but not midazolam (7B right panel).

FIG. 8A-8F show longitudinal stability of local field potential (LFP) recordings. A: average amplitude spectra across all intracranial channels collected 1 yr apart in first (black) and second (red), respectively. B: fold change of frequency-resolved amplitude between 2023 and 2024 with standard deviation across channels. C and E: auditory-evoked field strength for channels in auditory and motor cortex as a function of sound intensity. D and F: signal-to-noise ratio of averaged auditory-evoked potentials for channels in auditory and motor cortex as a function of sound intensity.

FIG. 9 shows a flow diagram of a method for measuring LFPs in a brain of a patient using a mesoscopic electrophysiological system, according to some non-limiting embodiments.

FIG. 10 shows a flow diagram of a method for monitoring a plurality of LFPs within a brain of a patient, according to some non-limiting embodiments.

FIG. 11 shows a diagram of example components of one or more devices of a mesoscopic electrophysiological system, according to non-limiting embodiments of the present disclosure.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal” and derivatives thereof shall relate to the present disclosure as it is oriented in the drawing figures. However, it is to be understood that the present disclosure may assume various alternative variations and step sequences, except where expressly specified to the contrary. Hence, specific dimensions and other physical characteristics related to the embodiments disclosed herein are not to be considered as limiting.

As used herein, the term “patient” is any mammal, including primates, and a “human patient”is any human.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, the terms “communication” and “communicate” refer to the receipt, transmission, or transfer of one or more signals, messages, commands, or other type of data. For one unit or device to be in communication with another unit or device means that the one unit or device is able to receive data from and/or transmit data to the other unit or device. A communication can use a direct or indirect connection, and can be wired and/or wireless in nature. Additionally, two units or devices can be in communication with each other even though the data transmitted can be modified, processed, routed, etc., between the first and second unit or device. For example, a first unit can be in communication with a second unit even though the first unit passively receives data and does not actively transmit data to the second unit. As another example, a first unit can be in communication with a second unit if an intermediary unit processes data from one unit and transmits processed data to the second unit. It will be appreciated that numerous other arrangements are possible. Any known electronic communication protocols and/or algorithms can be used such as, for example, TCP/IP (including HTTP and other protocols), WLAN (including 802.11a/b/g/n and other radio frequency-based protocols and methods), analog transmissions, Global System for Mobile Communications (GSM), 3G/4G/LTE, BLUETOOTH, ZigBee, EnOcean, TransferJet, Wireless USB, and the like known to those of skill in the art.

As used herein, “electrical communication,” for example in the context of transmitting electrical pulses from a pulse generator to an electrode refers to sending an electrical pulse produced by a pulse generator to an electrode, an electrode lead, a magnetic coil, or like devices capable of generating electrical current to stimulate a nerve or neuron as described herein, typically through an electrically conductive lead.

The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The terms “treat,” “treating”, “treatment,” “ameliorate” or “ameliorating” and other grammatical equivalents as used herein, can include alleviating, or abating a disease or condition symptoms, inhibiting a disease or condition, e.g., arresting the development of a disease or condition, relieving a disease or condition, causing regression of a disease or condition, relieving a condition caused by the disease or condition, or stopping symptoms of a disease or condition.

The term “preventing” can mean preventing additional symptoms, ameliorating or preventing the underlying metabolic causes of symptoms, and can include prophylaxis.

In some instances, “treat,” “treating”, “treatment,” “ameliorate” or “ameliorating” and other grammatical equivalents can include prophylaxis. “Treat,” “treating”, “treatment,” “ameliorate” or “ameliorating” and other grammatical equivalents can further include achieving a therapeutic benefit and/or a prophylactic benefit. Therapeutic benefit can mean eradication of the underlying disease being treated. Also, a therapeutic benefit can be achieved with the eradication of one or more of the physiological symptoms associated with the underlying disease such that an improvement can be observed in a subject notwithstanding that, in some embodiments, the subject can still be afflicted with the underlying disease.

The terms “effective amount”, “therapeutically effective amount” or “pharmaceutically effective amount” as used herein, can refer to a sufficient amount of a compound being administered which will at least partially ameliorate a symptom of a disease or condition being treated.

As used herein, the term “baseline local field potential (LFP)” means one or more local field potentials that may be detected with one or more of the plurality of electrode contacts after a system as described herein is affixed to a patient and one or more of the plurality of electrode contacts are inserted into the patient's brain. The term may also be used to refer to an LFP that is detected prior to an intervention, for example administration of a compound, such as a potential therapeutic, and/or a stimulation (e.g., electrical chemical, and/or mechanical) to the patient. As used herein the term “post-administration LFP” means one or more local field potentials that may be detected with one or more of the plurality of electrode contacts after an intervention, for example administration of a compound, such as a potentially effective amount of a therapeutic or a potential therapeutic, and/or a stimulation (e.g., electrical chemical, and/or mechanical) to the patient. A “post-administration LFP” includes LFPs detected from immediately prior to the intervention to 1 minute, 1 hour, 1 day, 1 week, 1 month, and/or 1 year following the intervention, all values and subranges therebetween inclusive.

### EXAMPLE

### Materials and Methods

The experiments were performed on one adult male macaque monkey (Macaca mulatta). The treatment of the monkey was in accordance with the guidelines set by the U.S. Department of Health and Human Services (National Institutes of Health) for the care and use of laboratory animals. All methods were approved by the Institutional Animal Care and Use Committee at the University of Pittsburgh.

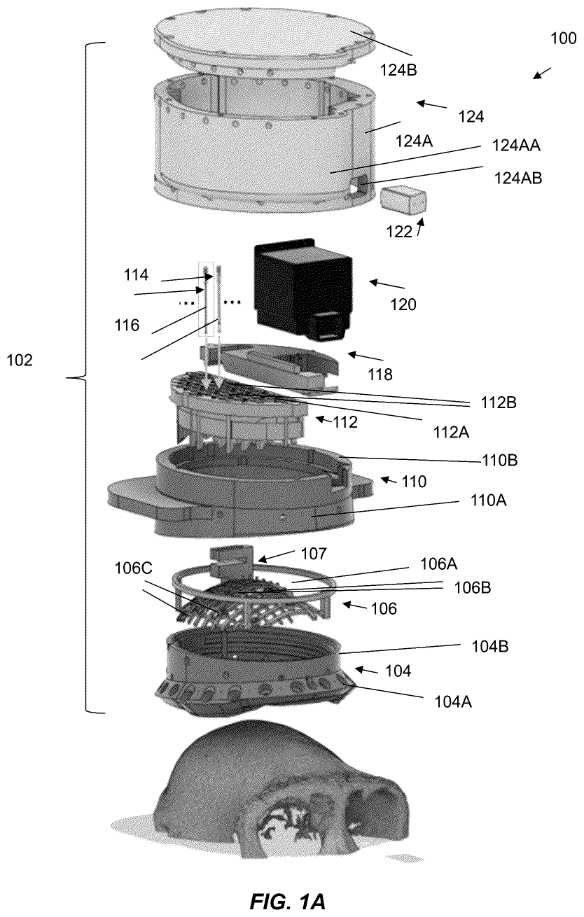

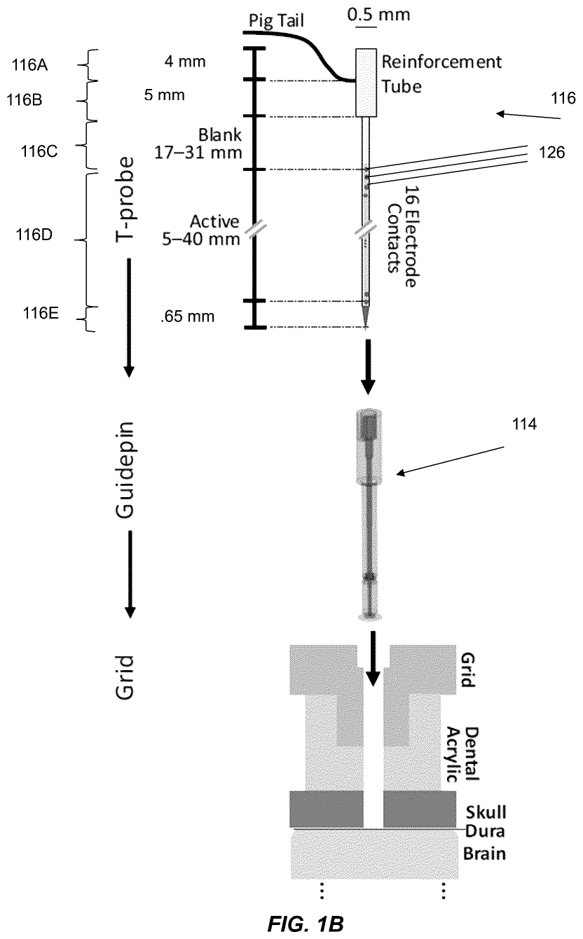

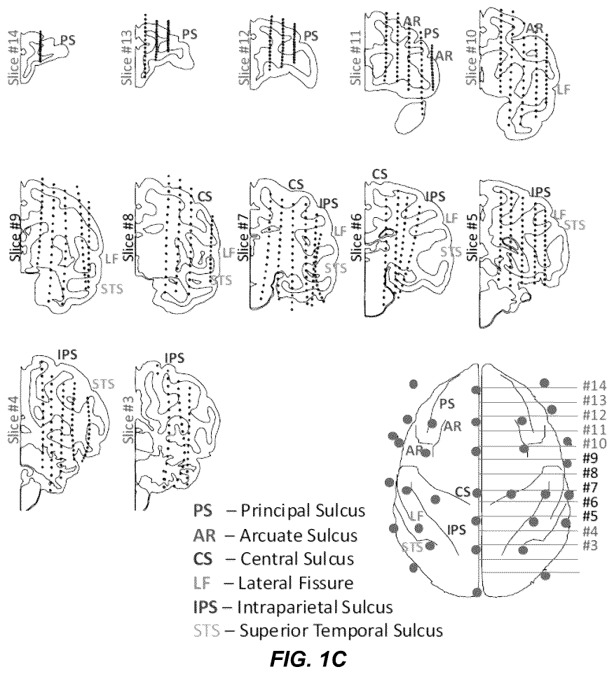

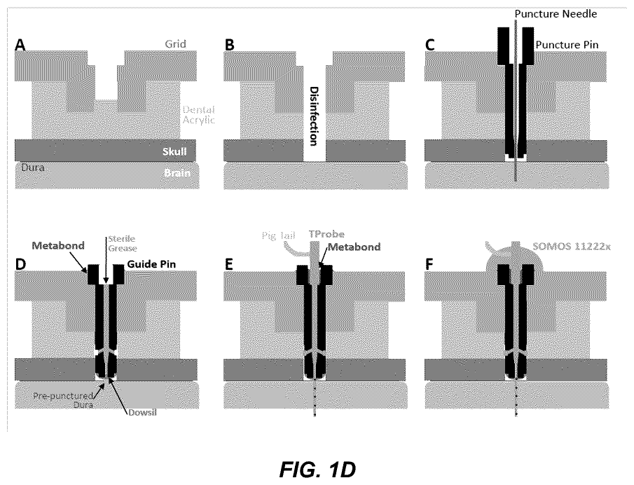

The MePhys platform consists of three rugged structural elements (“crown,” “wall,” and “cap”), two internal elements that facilitate the implantation of the macroscopic and mesoscopic electrodes (“spider” and “grid”), and the headstage (SpikeGadget) and its “baseplate”(FIGS. 1A-1C).

The MePhys platform may include mesoscopic electrophysiological system 100 for recording electrophysiological aspects of a brain of a patient. As shown in FIGS. 1A and 1B, mesoscopic electrophysiological system 100 may include structural system 102, a plurality of mesoscopic intracranial electrode shafts, and a plurality of guide pins sized and configured to receive the plurality of mesoscopic intracranial electrode shafts positioned therein.

In some non-limiting embodiments, structural system 102 may be sized and configured to be attached to a head of a patient and to hold a plurality of intracranial electrode shafts in predetermined positions around the head of the patient. As shown in FIG. 1A, structural system 102 may include crown component 104 (e.g., referred to as “crown”), semi-spherical grid component 106 (e.g., referred to as a “spider”), wall component 110 (e.g., referred to as “wall”), and grid component 112 (e.g., referred to as “grid”).

In some non-limiting embodiments, crown component 104 may include annular section 104A and top section 104B. In some non-limiting embodiments, crown component 104 is configured to be anchored to a skull of a patient via fasteners inserted through annular section 104A. In some non-limiting embodiments, semi-spherical grid component 106 may include first structure 106A having a first plurality of apertures 106B, wherein first structure 106A corresponds to a shape of a skull of the patient. In some non-limiting embodiments, semi-spherical grid component 106 may be positioned within crown component 104.

In some non-limiting embodiments, wall component 110 may include base section 110A and top section 110B. In some non-limiting embodiments, base section 110A may be configured to connect to top section 104B of crown component 104. In some non-limiting embodiments, grid component 112 may include second structure 112A that has a second plurality of apertures 112B. In some non-limiting embodiments, the second plurality of apertures 112B of grid component 112 are spaced apart in an antero-posterior direction and/or medio-later direction by a dimension in a range between 4 mm and 5 mm. In one example, the second plurality of apertures 112B of grid component 112 are spaced apart in an antero-posterior direction and/or medio-later direction by a dimension of 3 mm. In some non-limiting embodiments, one or more apertures 112B of the second plurality of apertures 112B has an inner diameter in a range between 1.0 mm to 2.0 mm. In one example, one or more apertures 112B of the second plurality of apertures 112B has an inner diameter that may be equal to 1.5 mm.

In some non-limiting embodiments, grid component 112 may be configured to fit within wall component 110 and/or may be configured to receive a plurality of guide pins 114 within the second plurality of apertures 112B. In some non-limiting embodiments, the second plurality of apertures 112B may align with at least a portion of the first plurality of apertures 106B to allow for a plurality of mesoscopic intracranial electrode shafts 116 to be inserted into the brain of the patient. In some non-limiting embodiments, guide pin 114 may include an interior channel, and a sterile grease may be provided in the interior channel of guide pins 114. In some non-limiting embodiments, guide pin 114 may include a vent connected to the interior channel, and the vent may provide a path for the sterile grease to travel when mesoscopic intracranial electrode shaft 116 is positioned within the interior channel of guide pin 114.

As further shown in FIG. 1A, structural system 102 may include cap component 124 that may include cap section 124A and cap top section 124B. In some non-limiting embodiments, cap section 124A may include wall 124AA that has peripheral aperture 124AB. Wall 124AA may define a main aperture, and cap section 124A may be configured to connect to top section 110B of wall component 110. In some non-limiting embodiments, cap top section 124B is configured to cover the main aperture of wall 124AA, and/or peripheral aperture 124AB may be sized and configured to allow for cables that connect to mesoscopic intracranial electrode shafts 116 to pass therethrough.

As further shown in FIG. 1A, structural system 102 may include baseplate 118 and headstage 120. In some non-limiting embodiments, baseplate 118 may be sized and configured to hold headstage 120 in an appropriate position. As further shown in FIG. 1A, structural system 102 may include plug 122 that is sized and configured to fit into peripheral aperture 124AB.

As further shown in FIG. 1A, mesoscopic electrophysiological system 100 may include a plurality of mesoscopic intracranial electrode shafts 116, which may include a first plurality of electrode contacts (not shown in FIG. 1A) configured to measure local field potentials (LFPs) at a plurality of locations in a brain of a patient. As shown in FIG. 1B, each electrode shaft 116 may include a second plurality of electrode contacts (e.g., a subset of the first plurality of electrode contacts). In some non-limiting embodiments, the first plurality of electrode contacts forms a three-dimensional grid within the head of the patient. In some non-limiting embodiments, the plurality of contacts (e.g., the first plurality of electrical contacts, a subset of the electrical contacts, etc.) may be configured to provide electrical micro-stimulation to a plurality of locations in the brain of the patient. In some non-limiting embodiments, the first plurality of electrode contacts may be configured to provide at least 1,000 channels for measuring LFPs at a plurality of locations in the brain of the patient. In some non-limiting embodiments, the first plurality of electrode contacts may be configured to measure the LFPs at the plurality of locations in the brain of the patient at a sampling rate in a range between 20 kHz and 30 kHz.

In some non-limiting embodiments, semi-spherical grid component 106 may include a plurality of electroencephalogram (EEG) electrodes 106C that are positioned to contact a surface of the head of the patient when structural system 102 is placed on the head of the patient. As further shown in FIG. 1A, mesoscopic electrophysiological system 100 may include EEG connector 107, to connect the plurality of EEG electrodes 106C to a processor (not shown in FIG. 1A) of mesoscopic electrophysiological system 100, so that EEG signals may be processed.

In some non-limiting embodiments, mesoscopic intracranial electrode shaft 116 may include a plurality of sections along a length of mesoscopic intracranial electrode shaft 116. As shown in FIG. 1B, mesoscopic intracranial electrode shaft 116 may include top tube section 116A, bottom tube section 116B, blank section 116C, active section 116D, and tip section 116E. In some non-limiting embodiments, top tube section 116A and bottom tube section 116B may have a length equal to a length of a reinforcement tube at a first end (e.g., an end that extends away from an end that penetrates the brain of the patient) of mesoscopic intracranial electrode shaft 116. In some non-limiting embodiments, top tube section 116A may have a length that starts at the first end and ends at a connection point of a cable (e.g., a “pig tail”) for measuring aspects of the brain. In some non-limiting embodiments, top tube section 116A may have a length in a range between 3 mm and 5 mm. In one example, top tube section 116A may have a length equal to 4 mm. In some non-limiting embodiments, bottom tube section 116B may have a length that starts at a connection point of a cable and ends at a point where the reinforcement tube meets a main body (e.g., a main body made up of blank section 116C, active section 116D, and tip section 116E) of mesoscopic intracranial electrode shaft 116. In some non-limiting embodiments, bottom tube section 116B may have a length in a range between 4 mm and 6 mm. In one example, bottom tube section 116B may have a length equal to 5 mm. In some non-limiting embodiments, the reinforcement tube may have a diameter in a range between 0.4 mm and 1 mm. In one example, the reinforcement tube may have a diameter equal to 0.5 mm.

In some non-limiting embodiments, blank section 116C may include a length of mesoscopic intracranial electrode shaft 116 that does not include a plurality of electrode contacts 126. In some non-limiting embodiments, blank section 116C may have a length in a range between 17 mm and 31 mm. In one example, blank section 116C may have a length equal to 20 mm. In some non-limiting embodiments, active section 116D may include a length of mesoscopic intracranial electrode shaft 116 that includes the plurality of electrode contacts 126. In some non-limiting embodiments, active section 116D may have a length in a range between 5 mm and 40 mm. In one example, active section 116D may have a length equal to 25 mm. In some non-limiting embodiments, a plurality of electrode contacts 126 may have variable inter-electrode spacing (e.g., spacing along a length of mesoscopic intracranial electrode shaft 116) in a range between 250 μm and 3.5 mm. In some non-limiting embodiments, a number of the plurality of electrode contacts 126 may be in a range between 10 and 24 electrodes. In one example, the number of the plurality of electrode contacts 126 may be equal to 16.

In some non-limiting embodiments, tip section 116E may include a length of mesoscopic intracranial electrode shaft 116 that is sized and configured to penetrate into the brain of the patient. In some non-limiting embodiments, tip section 116E may have a length in a range between 0.5 mm and 1.25 mm. In one example, tip section 116E may have a length equal to 0.65 mm.

With regard to the above description, the crown and wall were initially designed in house using Solidworks and then refined and manufactured by Graymatter from PEEK using a 5-degree-of-freedom CNC machine. The crown is the main structural element that is anchored to the skull with metabond and a ring of ceramic screws around the outer margin. It forms a tight seal between the soft tissue on the outside and the inside of the implant that consists of bone covered in metabond and acrylic. The wall is the second structural element that increases the height of the implant and provides the “wings” for head-fixation. The role of the spider and the grid is to determine the location of the EEG and intracranial electrode shafts, respectively. Both parts were designed in-house using Fusion360 and manufactured from SOMOS resin on a VIPER SLA printer. The top of the spider is the mounting point for the 32-channel Omnetics connector of the EEG electrodes. The arms of the spider guide the wires from the connector to the electrodes without getting in the way of the to-be-implanted depth electrodes. The holes in the grid are the mounting points for the guide pins and, thus, determine the locations of the mesoscopic electrode shafts. The holes were spaced out by 5 mm and 4 mm in the antero-posterior and medio-lateral direction, respectively. The inner diameter of each grid-hole was 1.5 mm. The baseplate provided a stable platform for the SpikeGadgets headstage and was designed to align the mini-HDMI connector with the corresponding opening in the cap. This allowed us to record from all channels without taking off the cap. The cap was 3-D printed with Nylon by Graymatter. In the latest iteration, the cap is split into two parts to provide easy and flexible access to the amplifier and the connectors without having to take off the entire cap. The total weight of the implant was estimated at ˜360 g (crown [symbol] wall: 120 g; grid: 20 g; baseplate: 10 g; Spider: 5 g; cap: 85 g; SpikeGadgets headstage: 20 g; we estimated another 100 g of dental acrylic). After a period of getting used to its presence, the implant is not causing any noticeable problems with the movement or behavior of the animal.

The guide pins are functional elements of the MePhys system. Their role is 1) to allow all 62 electrode shafts to penetrate the dura without buckling or breaking and 2) to subsequently keep bacteria or viruses from entering the brain along any of the electrode shafts. The guide pins were designed in-house in Fusion360 and manufactured from SOMOS resin on a VIPER SLA printer. The outer diameter of the guide-pins was 1.45 mm, allowing them to slide into the 1.5 mm holes in the grid. To help the electrodes penetrate the dura without buckling, the guide pins featured a very thin inner diameter (500 μm), that narrowed even further at the tip (335 μm). Although the diameter of the stainless steel tubes of the electrode shafts was 260 μm, the actual diameter of the finished electrodes was a bit larger. Similarly, the actual inner diameter of the printed guide pins was smaller than the nominal 335 μm. As a result, the bottom of the guide pins provided extremely close lateral support and prevented any lateral movement during penetration. Furthermore, the length of the pins was custom designed for each grid hole to lightly touch and dimple the underlying dura, thus facilitating the penetration. This was especially important for the most lateral shafts that had to penetrate the dura at an oblique angle. In addition, we also used puncture pins with identical dimensions to pre-puncture the dura in the exact location that the electrodes would later be inserted. Using this approach, all electrode shafts penetrated the dura without problems.

Several steps were taken to prevent bacteria or viruses from entering the brain along the electrode shafts or the outside of the guide pins. First, the guide pins were sterilized in a hydrogen peroxide sterilizer. Using fully sterile procedures, the sterilized pins were backfilled with sterile grease, and the tip was closed with a drop of KwikSil. Both approaches have been used successfully in the Graymatter SC system. Prior to being implanted, the prepared guide pins were again sterilized using hydrogen peroxide. To prevent bacteria or viruses from entering along the outside of the guide pins, they were cemented into the grid-hole during the surgery using metabond. The guide pins also featured an array of four small holes located 5 mm from the tip that allowed sterile grease to be pushed out of the guide pin when the electrode was inserted. This created a ring of sterile grease in the tight space between the inside of the grid hole and the outside of the guide pin.

Referring now to FIG. 1D, FIG. 1D shows an implementation of a procedure for applying a plurality of guide pins (e.g., guide pins 114) to a head of a patient using a grid component (e.g., grid component 112) of mesoscopic electrophysiological system 100.

As shown in A of FIG. 1D, the grid component may be applied to the skull of the patient using a dental acrylic to bond the grid component to the skull. As shown in B of FIG. 1D, a hole may be drilled into the dental acrylic that corresponds to an aperture in the grid component and the hole may be disinfected using a disinfectant. As shown in C of FIG. 1D, a puncture pin with a puncture needle may be inserted into the hole, and the puncture needle may be used to puncture the dura and the brain of the patient. As shown in D of FIG. 1D, the puncture pin may be removed from the hole, and a guide pin may be inserted into the hole and held in place in an amount of dural sealant at a bottom end of the guide pin. An interior channel of the guide pin may be filled with a sterile grease. As shown in E of FIG. 1D, a mesoscopic intracranial electrode shaft may be inserted into the interior channel of the guide pin so that a plurality of electrode contacts are positioned into the brain of the patient. As the electrode shaft is inserted into the interior channel of the guide pin, the sterile grease may be displaced into a vent on either side of the interior channel. Additionally, a reinforcement tube of the electrode shaft may be held in place in the guide pin with an adhesive. As shown in F of FIG. 1D, a cable connected to the electrode shaft may be connected to a processor of mesoscopic electrophysiological system 100 to provide signal measurements (e.g., measurements of LFPs at various locations in the brain).

The MePhys system was implanted in a series of five surgeries distributed across a period of 8 months. All surgeries were performed under the highest standards of sterility, given that it would be impossible to treat any infection without removing the entire implant. The goal of the first surgery was to implant the MePhys platform and the EEG electrodes. Prior to the surgery, we prepared the EEG electrodes and the Omnetics connector and fastened them to the spider. Both spider and EEG connector were mounted into the crown and sterilized. During the surgery we exposed the skull, removed any residual tissue, and sealed the pores in the bone with copalite varnish. Crown and spider were positioned on the skull using a stereotactic arm and cemented in place with Metabond. Next, the holes for the ceramic screws were drilled and taped. The ceramic screws were then fastened into the holes around the outside diameter of the “crown.” A second layer of Metabond was applied from inside and outside of the “crown” to create a tight seal. We then drilled the holes for the EEG electrodes into the skull and cemented them in place with small drops of Metabond. Additional ceramic screws were then added in free spaces over the left hemisphere to increase the connection between skull and implant. Next, we installed the wall and fixed it in place with set-screws. The gaps between the “crown” and “wall” were filled with a thin layer of Dowsil that had been applied prior to combining the two elements. The grid was positioned over the right hemisphere and cemented in place by filling the space underneath it with dental acrylic. The ceramic screws over the left hemisphere were covered with additional layers of dental cement. After inserting the “baseplate” over the left hemisphere, the implant was covered with a flat cap before recovering the animal.

After the initial surgery, a computed tomography (CT) image was collected to determine the actual location of the platform and the exact locations of the grid holes. Based on this image, we determined that 58 of the 72 grid holes were suitable for electrode implantation. 11 of the grid holes were excluded because they were closer to the anterior cerebral artery in the longitudinal fissure than intended. The two grid holes in the most posterior slice were excluded because drilling through them might have damaged some of the EEG wires that seemed to have moved around during the surgery. One grid hole was excluded because it was near a fault in the underlying dental acrylic, which raised concerns about sterility. The electrode shafts were implanted over the course of the next four surgeries. Two shafts were implanted during the first electrode implant surgery. As we improved our implantation technique over time, we implanted 4, 21, and 31 electrode shafts during the next three surgeries.

Because the back-ends of already implanted electrode shafts cannot be sterilized in place, it was increasingly more challenging to create a sterile field for subsequent surgeries. Our solution was to unplug the connectors of all previously implanted electrodes and cover them under a sterile custom designed cap. This approach succeeded at creating a sterile field, but we lost functionality of several electrodes in the process of repeatedly unplugging and re-plugging them before and after surgeries.

To implant an individual depth electrode into a grid-hole, we first drilled through the underlying acrylic and bone. We then pre-punctured the dura using a puncture pin and cemented a custom-designed guide pin in place that lightly touched and dimpled the underlying dura. Next, we inserted the electrode shaft into the guide pin and slowly pushed it through the dura and into the brain by hand until it reached its final position inside the guide pin. Finally, the top of the guide pins and the shaft of the electrodes were covered with 3-D resin that was polymerized in place with an ultraviolet (UV) lamp during surgery. Since the grid had been printed with the same resin, this provided an extremely tight and stable seal.

The increase in the number of shafts implanted in successive surgeries was achieved by designing better surgical tools and workflows. Most importantly, we created a set of 25 3-D printed drill-bit spacers in 1-mm increments. Based on the MRI image acquired after implanting the base, we identified the correct spacer for each grid hole prior to the surgery. We also transitioned from a manual to an electric drill to advance the hole to within 1 mm of the dura. Only the last 1 to 2 mm of the holes were then drilled by hand.

Of the 57 electrode shafts that we implanted, 56 were fully functional after the respective surgery. However, 6 of them subsequently lost functionality due to mechanical problems with the electrode backend. In four cases, we can link the loss of signal to plugging and unplugging of the connector that was necessary for subsequent surgeries. In two instances, the exposed tops of the electrodes with the pig-tails ripped off. Other than these instances of mechanical failure, we have not noticed any degradation of the recorded LFPs over time. As expected, we did lose isolated single cells over the first couple of months. However, we can still record stable multiunit activity on many of the same channels more than 4 months after implantation.

The MePhys system is built around the 1,024 channel modular headstage from SpikeGadgets (SpikeGadgets LLC). The headstage is small and light enough to be permanently enclosed in the MePhys platform on the left side of the animal's head. It provides easy access to all 1,024 channels via a single micro-HDMI connector at a sampling rate of 20 or 30 kHz. The SpikeGadgets system also allows untethered recordings, a feature that we plan to take advantage of soon. The system consists of a maximum of 8 modular amplifier boards each supporting 128 channels via two 64-channel ZIF connectors. The T-Probes were connected to the headstage using flexible printed circuit board (PCB) cables that were designed in house and manufactured by PCBWay. The flex-PCB cables featured a 64-channel ZIF connector on one end that branched into four narrower cables, each with a female 18-channel Omnetics connector that mated with the male Omnetics connectors of the T-Probes. The exact layout of each of the 16 flex PCB cables was designed in Fusion360 to each match the location of a different set of four of the 62 planned electrode shafts. This custom design of the backend was critical to enable a coherent design of the whole system, including the implantation grid, the location of the headstages, and the management of the flexible cables.

We designed our MePhys system to combine 32 macroscopic EEG electrodes with 992 intracranial electrode contacts distributed across 62 electrode shafts arranged on a regular grid in the horizontal plane over the right hemisphere. The grid was arranged into 14 coronal slices and featured between 2 and 7 grid holes per slice. Based on CT and MRI images, we customized the electrode shafts for each grid hole to traverse the underlying Tel-, Met-, and Diencephalon, and parts of the Mesencephalon, but excluding the pons and medulla, and avoiding the Circle of Willis. The 16 electrode contacts on each shaft were spaced out regularly across the entire section of brain parenchyma traversed by the electrode shaft. The EEG electrodes were positioned in an approximately regular two-dimensional (2-D) array on the skull surface that was designed using our monkey 10-20 software package. EEG electrode locations on the right hemisphere were adjusted slightly so as not to interfere with the location of the intracranial electrode shafts.

To confirm the intended locations of the electrode shafts and contacts, we acquired a postsurgical CT scan with all the implanted electrode shafts in place. In most cases, the electrode shafts were close to their intended locations. We observed a slight slant in some shafts which we believe was caused by minor pre-existing bends in the stainless-steel tubes. Two electrode shafts seemed to be entering the brain at an oblique angle, suggesting that the holes through the acrylic had not been drilled perfectly perpendicularly to the grid. This is likely the result of these holes being drilled by hand. There were also some subtle discrepancies in the vertical position of the probes. These vertical discrepancies arose from the fact that all electrode shafts were between ˜3 mm and 13 mm longer than specified, due to a miscommunication with the manufacturer. This discrepancy was only noticed after the third electrode implant surgery and caused the affected electrodes to penetrate deeper than intended. In four extreme cases, the additional length caused the electrode shafts to come into contact with the bone on the ventral side of the skull and buckle. However, in most instances, the electrode contacts just ended up a bit deeper than originally intended, causing the topmost contacts to be below the dorsal cortex, rather than above it. Once we became aware of the problem, we corrected it for the additional length by adding a custom designed stopper in-house. This improved the match between intended and actual electrode locations for the 31 shafts implanted in the last surgery. However, the stopper tended to overcompensate and left some of the topmost electrode contacts outside of the brain parenchyma. Unfortunately, the mechanical link between stopper and electrode shaft was weaker than for the original reinforcement tube in some cases. In combination with the electrode shafts extending up to 13 mm higher above the grid than intended, this left the electrode back-ends more vulnerable forces generated by rapid head-movements and caused two of them to break off. We have since mechanically stabilized the remaining shafts using Loctite Foam.

Following each implant surgery, we followed our standard postsurgical care protocol that consists of Baytril (5 mg/kg SID) for 5 days, Cartofen (4.4 mg/kg once a day) for 2 days, and dexamethasone (0.2 mg/kg twice a day) for 3 days. The average weight of the animal around the time of the surgeries was 14.5 kg. In the 1-2 weeks following each surgery, we observed a small temporary drop in weight on the order of 0.3 and 0.5 kg. Currently, ˜18 months after the last surgery, the animal weighs 16.3 kg.

After optimizing our surgical approach, we were able to implant 31 shafts in the last surgery (no. 5). The animal was sitting up within 5 min. of returning to the home cage. Subsequent behavioral monitoring in the home-cage identified no postsurgical impact on motor function, appetite, or demeanor. We believe that this fast recovery is the normal outcome of a MePhys surgery even if many shafts are implanted. However, in earlier surgeries, we encountered two adverse events that complicated postsurgical recovery. During the second surgery (two shafts implanted), a problem in the anesthesia setup (malfunctioning scavenger system) presumably increased the isoflurane concentration beyond the intended levels. This caused the animal to emerge from anesthesia much slower than usual and seemed to have reduced his appetite. We treated the animal with cerenia (0.5 mg/kg one-time dose) for nausea and extended the duration of the dexamethasone for an additional day. Based on behavioral observation, the animal fully recovered within 48 h. During the fourth surgery (21 shafts implanted), some of the shafts were implanted deeper than intended and buckled (see Electrode Locations). This buckling caused noticeable motor deficits in the contralateral arm and foot. We extended dexamethasone for a total of 10 days after surgery. The motor deficits took ˜2-4 weeks to resolve.

Details of the cranial EEG recordings have been reported previously. Briefly, 33 EEG electrodes manufactured in-house from medical grade titanium were implanted in 1-mm deep, nonpenetrating holes in the cranium. The electrodes were connected to a 36-pin Omnetics connector mounted in the left posterior aspect of the MePhys platform. The 33 EEG electrodes formed an approximately regularly spaced grid on the skull covering roughly the same anatomy covered by the international 10-20 system. The position of EEG electrodes on the right hemisphere were adjusted to fall in between the grid holes for the intracranial electrode shafts.

The intracranial probes (T-Probes) were custom designed and hand-made multi-contact probes (NeuronElektrod Kft, distributed by Plexon Inc.). The dimensions of the T-Probes were determined based on a CT scan acquired after the implantation of the MePhys platform. Based on this CT image, we determined individual electrode specifications for each of the 62 grid holes based on the underlying anatomy. Two main parameters determined the layout of each electrode. 1) The length of the active portion of the shank toward the tip of the probe “a,” and the blank or passive portion toward the top of the probe “b” (FIG. 1B). The length of the blank portion of the probe was designed to traverse the distance from the surface of the grid to the inner surface of the dorsal aspect of the skull. The length of the active portion of the probes was chosen to traverse the entire underlying brain tissue, including Tel-, Met-, and Diencephalon, and parts of the mesencephalon, but excluding the pons and medulla, and avoiding the Circle of Willis. The 16 electrodes were spaced uniformly across the active portion of the shank, thus determining the interelectrode spacing. Depending on the length of the active portion, interelectrode distance along a shaft ranged between 2.7 mm and 0.4 mm. The very top of each shank contained a 9-mm long reinforced section with a pig-tail connector that terminated in an 18-channel Omnetics connector.

From a technical point of view, T-probes are a modification of the popular V- and S-Probe. The requirement of site spacing was to be equidistant across the entire active portion of the shank, rather than the standard predefined 30 μm, 50 μm, or 75 μm, etc. Since the active portion of the shank was different for each probe, the required interelectrode spacing also differed for each probe. The key challenge of the T-Probes was to accommodate the large interelectrode spacing that required the opening in the stainless steel tube to be considerably larger than in the standard V-and S-Probe and still keeping suitable strength and flexibility of the shaft.

All experiments were performed in a small (4 ft wide by 4 ft deep by 8 ft high) sound-attenuating and electrically insulated recording booth (Eckel Noise Control Technology). The animal was positioned and head-fixed in a custom-made primate chair (Scientific Design). Neural signals were recorded with a 1,024-channel digital amplifier system (SpikeGadget) at a sampling rate of 20 kHz.

Experimental control was handled by a windows PC running an in-house modified version of the Matlab software package “monkeylogic”. Sound files were generated prior to the experiments and presented by a subroutine of the Matlab package “Psychtoolbox.” The sound-files were presented using the right audio-channel of a high-definition stereo PCI sound card (M-192 from M-Audiophile) operating at a sampling rate of 96 kHz and 24-bit resolution. The analog audio-signal was then amplified by a 300 W amplifier (QSC GX3). The amplified electric signals were converted to sound waves using two single element 4 in. full-range driver (Tang Band W4-1879) located 8 in. in front of the animal to the left and right of the screen.

The resting state paradigm consisted of ˜3-5 min. long blocks during which the animal was not required to perform any task and during which no external stimuli were presented. During that time, eye-position and pupil diameter were monitored with an infrared eye-tracker. Resting-state data sets analyzed here were recorded before and after injection of subanesthetic doses of ketamine (1.9 mg/kg) and midazolam (0.39 mg/kg). In addition, we analyzed two resting state data sets recorded right before and after the animal fell asleep. These two recordings were made in the evening (6:30 PM) and night-time (11:00 PM). All other recordings were made in the context of his normal work routine that typically extends from late morning (10:00 AM) to early afternoon (1:30 PM).

In the RFmap noise paradigm, animals passively listened to brief 25-ms long pulses of white noise with a 5 ms linear rise/fall time. The white noise bursts were presented at five intensity levels (46, 56, 66, 76, 86 dB sound pressure level; SPL). Stimulus onset intervals ranged between 300 and 400 ms. The RFmap noise data sets analyzed here were recorded before and after injection of subanesthetic doses of ketamine (4.5 mg/kg) and midazolam(0.48 mg/kg).

The delayed frequency discrimination paradigm has been described in detail before. Briefly, the task measured the animals'ability to discriminate the tonal frequency of sequentially occurring pure tone pips. Each trial consisted of up to 13 tones (60 dB SPL, 205-ms duration, 5 ms rise and fall time). 80% of trials contained one target tone of different frequency relative to the preceding standard tones (target-present trials). In the remaining 20% of trials, the “target” tone was identical to the standard (catch trials). Animals moved a lever off-center to initiate a trial. On catch trials, animals were rewarded for not releasing the lever during the entire trial. In the “target-present trials,” they were rewarded for releasing the lever between 100 and 800 ms after target onset. For the analyses presented here, we focused on the trials in which the animal released the lever and the same number of matched control trials without a lever release.

Time-continuous raw data was filtered in MATLAB (v.2019a) using a 256 point noncausal digital low-pass FIR filter (firws function from EEG-laboratory toolbox, Blackman Window, high-frequency cutoff: 400 Hz). The filtered data was then cut into short epochs around external events, such as sound onset. The epoched data were then downsampled from 20 to 1 kHz and saved as MATLAB data files. In the case of the resting state recordings that contained no external events, we split the data into continuous 1-s long chunks that could either be analyzed as 1-s long trials or concatenated to recover the time-continuous traces.

The main analyses were performed using in-house analysis software package implemented in R (v.4.3.3). After reading data from all channels, we computed the averaged auditory-evoked potentials as a function of the five different sound pressure levels. Time-resolved auditory-evoked potentials were visualized in multi-plots in which the relative position of each panel corresponded approximately to the relative position of the channels in each slice. For visualization purposes, the potentials at EEG electrodes were scaled by a factor of 2. In addition, we presented data in the form of heat plot panels that visualized activity of the intracranial electrode contacts distributed across 12 coronal slices and the activity of the EEG electrodes distributed over the surface of the skull.

After loading the epoched LFP data of all functional channels, the data was re-referenced to the common reference of all intracranial channels. LFP amplitude spectra were computed using fast Fourier transformation in each of the 1-s long epochs using a 25-ms long cosine window taper. The spectra were then averaged across all trials. From the averaged spectra, we extracted the dominant frequency as the frequency with the highest amplitude. The dominant frequency was then color coded and displayed as heat-maps.

The epoched data were concatenated into a ˜3-5 min. long time-continuous version. The time-continuous data were bandpass filtered to extract activity in five frequency bands: low (0.1-1 Hz, 1st order Butterworth filter), δ (2-4 Hz, 3^{rd} order), θ/(low) α (5-10 Hz, 3rd order), (low) β (10-20 Hz, 3^{rd} order), and γ (20-40 Hz, 3rd order). Envelopes were computed by full-wave rectifying the band-passed signals and band-pass filtering the resulting time-series with a first-order Butterworth filter: δ envelope (0.1-1 Hz), θ/low α envelope (0.1-5 Hz), β envelope (0.1-8Hz), and γ envelope (0.1-16 Hz). We also created low-pass versions of the envelopes using the same rectification procedure but replacing the band-pass filters with a first order 0.1 Hz low-pass filter, regardless of carrier frequency. All envelopes and low-pass envelopes were then re-referenced to the average of all intracranial channels and then z-scored within each channel. We also kept a second version of the data that was not z-scored. This non-normalized data set was used to extract representative time courses for brain regions (see Extracting activity of a brain region). The envelopes were downsampled to 100 Hz, and the low-pass envelopes were downsampled to 10 Hz to reduce memory and CPU demands. The sampling rate of the band-passed data was maintained at 1,000 Hz.

Seed-based functional connectivity maps for a particular data set were computed by calculating the temporal correlation of all channels with that of a specific seed channel or seed region. Similarly, the functional connectomes were computed as the temporal correlation matrix of all channels. Principal components of all connectomes were computed with the princomp function in R.

To extract activity that is representative of a particular brain region, we first identified all channels in that brain region and then used the fastICA function in R to identify the time course of the first principal component. Two slightly different variants of the approach were used. The first version removed the mean across all channels in the region prior to computing the first principal component and the second one did not. The latter approach was used as a default; the former case was used when extracting and comparing multiple regions that may be subject to a shared volume-conducted signal.

We aimed to identify the emergence and recurrence of stable spatial patterns by computing spatial correlations between different points in time during a resting-state recording. A complete description of the similarity of spatial patterns over time is provided by the spatial correlation matrix. It is related to the temporal correlation matrix that gives rise to the functional connectome, but instead of identifying channels with similar time-courses, it identifies time-points with similar patterns of activity across all channels. In the simplest case, the spatial pattern can be computed from a single data set, such as the low-passed delta envelopes. However, it can also be extended to include more than one data set. We had identified the four low-pass envelopes as the most likely substrate for the emergence of intrinsic states. Hence, we concatenated the four data sets along the channel dimension, thus quadrupling the number of channels and calculated the spatial correlation matrix for this concatenated data set. Two time-points will show strong correlations if they have similar spatial patterns for all four low-passed envelopes. The emergence of stable states would be indicated by high spatial correlations for a contiguous period. A stable state would visually be noticeable as a region of increased width on the main diagonal of the spatial correlation matrix. The recurrence of a stable state should then be visible as a region of increased spatial correlations off of the main diagonal. If the states themselves are stable and the transitions between states are quick, then the regions of increased and decreased spatial correlations will be approximately square-shaped.

Longitudinal stability was analyzed using two separate metrics: 1) amplitude spectra during rest and 2) amplitude of auditory-evoked potentials. In both cases, we compared data collected in August and September of 2023 to data collected one year later in August and September of 2024. We only included data sets on days without drug injection. Furthermore, we only included the first resting-state data set collected at the beginning of the daily recording session; separate preliminary analyses had identified very pronounced and systemic differences between resting-state data sets collected at the beginning and the end of the daily recording session. We identified 38 and 23 resting-state data sets recorded in 2023 and 2024, respectively. For each recording session, we extracted 1-s long continuous epochs and computed fast Fourier transforms for each of them. The amplitude spectra was then averaged across all epochs in a session. We next averaged the amplitude spectra for all sessions of a given year for each channel. We quantified the change across the two years by dividing the averaged amplitude measured in 2024 by the one measured in 2023 for each channel and each frequency. The frequency-resolved values of fold change were then averaged across all SEEG channels and converted using a log 2 transformation.

We identified 40 and 32 RFmap noise data sets from 2023 and 2024, respectively, that matched our inclusion criteria. We calculated the average auditory-evoked potentials for each of these data sets for all five intensity levels separately. We then identified all channels in and around primary auditory cortex and computed the average evoked field strength as the standard deviation across all channels and all timepoints in the range between 0 and 70 ms after tone onset. To correct for small differences in the total number of valid trials collected on different days, we only included the first 100 trials from each condition. We defined a corresponding value of field strength at baseline using the time-period from 140 to 70 ms prior to tone onset. Baseline correction was performed on the interval from 70 to 0 ms prior to tone onset, thus equidistant from the baseline and signal period. Signal-to-noise ratio was defined as the ratio of the square of the field strength during tone presentation and baseline. The same analysis was also performed for channels in and around motor cortex, defined as F1 and F2 of the D99 atlas.

### RESULTS

Designing and implanting a MePhys system with the desired features posed a number of technical challenges. 1) We needed to custom design and manufacture 62 laminar depth probes with variable lengths and interelectrode spacings and a recording stability spanning months to years. 2) It was crucial to design a guide-tube system that would a) allow all 62 electrode shafts to penetrate the dura without buckling or breaking, and b) subsequently keep infections from entering the brain along any of the electrode shafts. 3) Because the large number of electrode shafts could not all be implanted in a single surgery, we needed to design a physical platform that remained a) accessible to us over the course of successive surgeries, and b) secure from the monkey. 4) To relate the mesoscopic to macroscopic recordings, the system had to accommodate the implantation of EEG electrodes in the skull. 5) Finally, because it takes hours to connect such a large number of electrodes, they needed to remain permanently connected to the headstage. Hence, the platform needed to provide enough space on the animal's head for both the headstage and the wires connecting the stage to the electrodes. We designed the MePhys platform to solve these challenges by combining three rugged structural elements (“crown,” “wall,” and “cap”), two internal elements that facilitate implantation of the macroscopic and mesoscopic electrodes (“spider” and “grid”), and a 1,024-channel headstage (SpikeGadget) and its “baseplate” (FIG. 1A; MePhys Platform for details). A computed tomography (CT) image collected after implanting the MePhys platform (MePhys Platform Surgery) determined that 58 grid holes were suitable for electrode shaft implantation (Section 4.5). Over the course of the next four surgeries, we successfully implanted 57 electrode shafts, 50 of which remained fully functional through all surgeries and to this day, i.e., between 8 and 14 months as of March 2024 (FIG. 1B). A CT image collected after all surgeries allowed us to visualize trajectories of all electrode shafts and locations of all 800 functional electrodes (FIG. 1C).

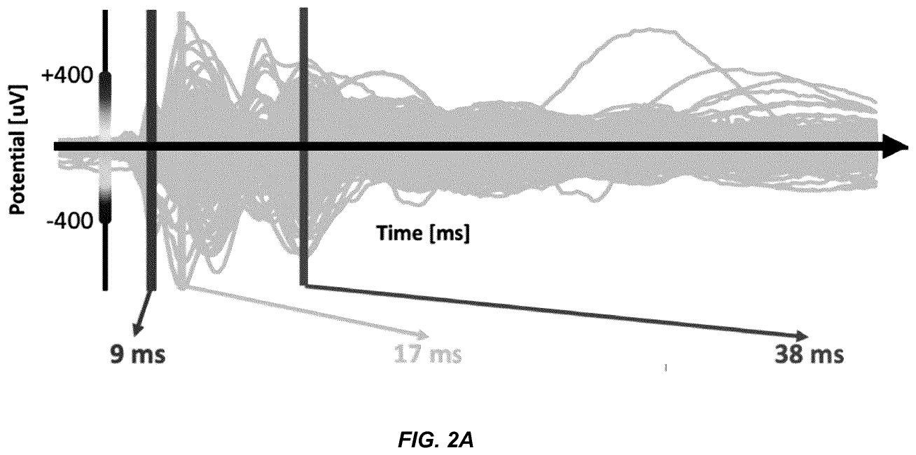

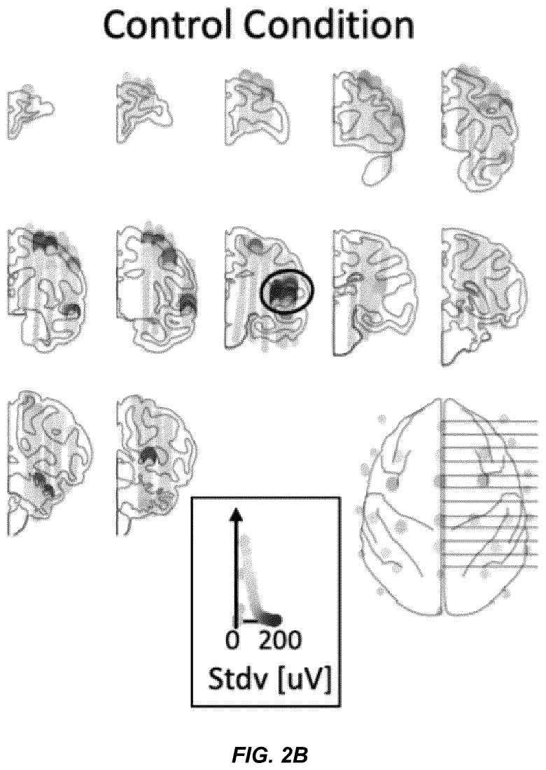

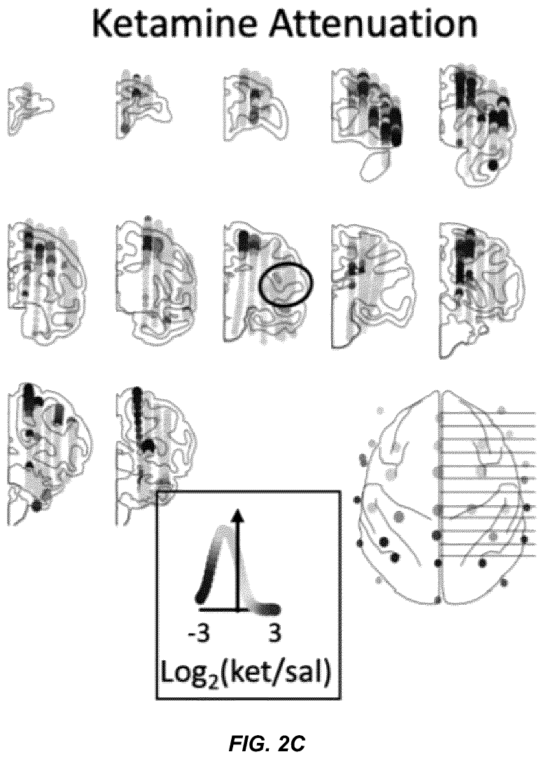

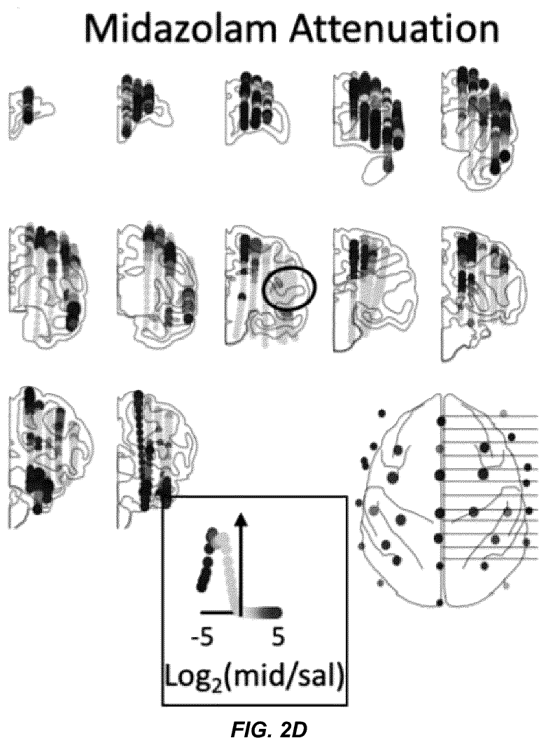

To showcase the ability of MePhys to track hemisphere-wide stimulus-evoked activity, we recorded electric activity from macro-and mesoscopic electrodes in response to short white-noise bursts of varying intensity at three key timepoints of the response (FIG. 2A). We were able to simultaneously record auditory-evoked electric fields (AEFs) from regions in the canonical ascending auditory pathway, including brainstem, inferior colliculus, thalamus, and a dense cluster of regions in and around primary auditory cortex (FIGS. 2A-2B). In addition, we also observed auditory-evoked responses in the insula; cerebellum; and prefrontal, posterior parietal, and motor cortices. The MePhys data allowed us to quantify evolving functional properties along the ascending auditory pathway, such as response latency, duration, and dependence on stimulus amplitude (data not shown). Consistent with earlier work (22, 47, 61, 62), we identified dipole-like electric fields in and around auditory and motor cortex that are believed to contribute to auditory-evoked EEG responses at the scalp, validating our system (FIGS. 2A-2B). Our results go beyond earlier work by simultaneously capturing electric fields across the entire hemisphere, rather than one-or two-dimensional partial slices. It is lasting 2-3 h. and featuring several active and passive paradigms. Hence, the data and analyses highlighted here represent only a tiny fraction of the available data and focus more on the breadth of possible analyses, rather than an in-depth analysis of one specific topic.

We next studied how the meso-and macroscopic auditory evoked responses are impacted by a subanesthetic dose of ketamine that is believed to mimic certain aspects of psychosis. To that aim, we recorded auditory-evoked responses 5 min. before and between 5 and 10 min. after ketamine injection. Our results confirmed earlier work that ketamine broadly attenuates auditory-evoked EEG responses. By simultaneously recording auditory-evoked fields across the entire hemisphere, we were now able to pinpoint these reductions to prefrontal, posterior parietal, and motor cortex, and cerebellum (FIGS. 2B-2C). Interestingly, the effects of ketamine on auditory cortex itself were minimal, with no reduction in overall response amplitude, and only subtle changes in latency of peaks and troughs. This suggests that ketamine either attenuated the propagation of auditory information from auditory cortex to downstream regions or selectively attenuated the responsiveness of all regions other than auditory cortex. We then compared the effects of ketamine to those of midazolam, which also attenuates auditory-evoked EEG responses, but does not mimic aspects of psychosis. In contrast to ketamine, midazolam led to strong attenuation of responses in auditory cortex. In addition, even stronger reductions of auditory-evoked responses were observed across the entire hemisphere and responses in other cortical regions and cerebellum were almost eliminated (FIG. 2D).

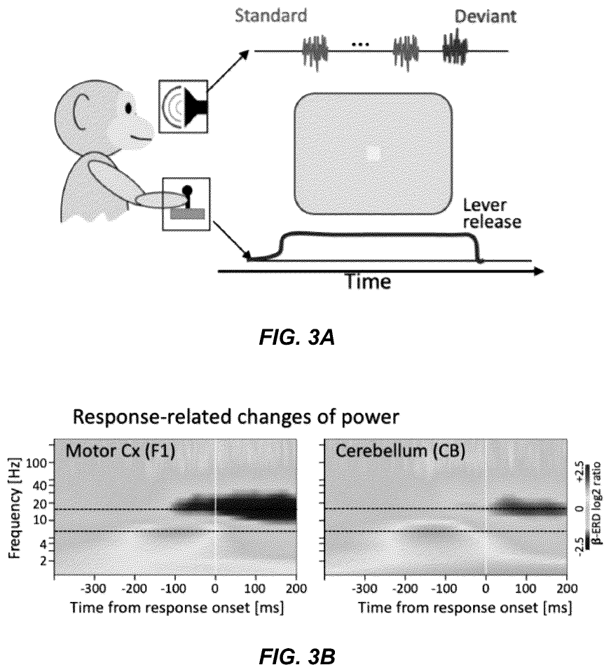

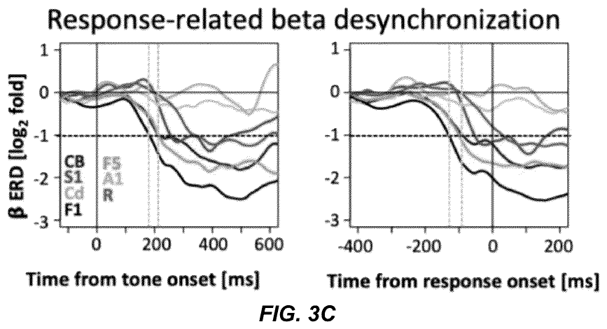

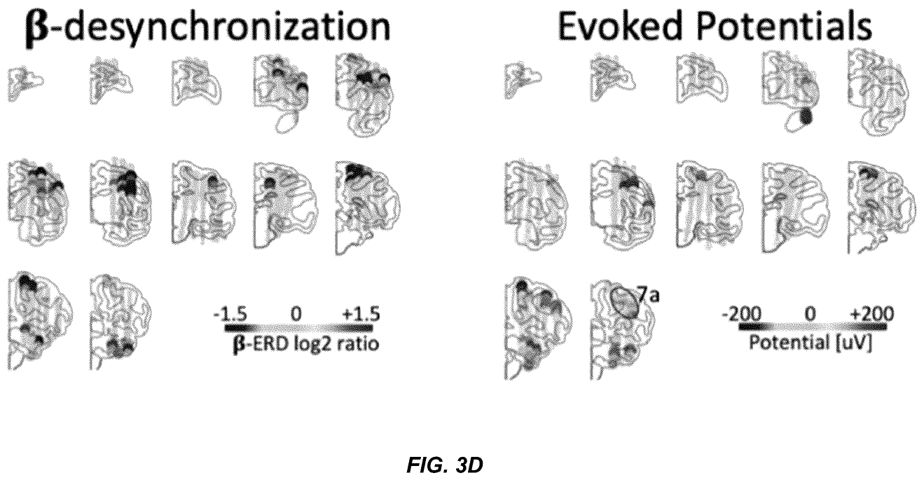

To probe the ability of MePhys to measure hemisphere-wide motor signals, we used an auditory change detection task during which the animal released a lever when it detected a deviant target tone (FIG. 3A). Strong movement related 18 Hz β-band desynchronization is visualized for two example regions, cerebellum (CB) and motor cortex (F1) (FIG. 3B). FIG. 3C shows the time-course of movement related b desynchronization for seven brain regions in the auditory (A1, R), motor (F1, F5, Cd), and somatosensory system (S1). The strongest and earliest β-band desynchronization was observed in motor cortex, followed 25 ms later by cerebellum and supplementary motor cortex (FIG. 3C). FIG. 3D compares the timing and spatial distribution of the β-band desynchronization with that of the response-related evoked potentials. Though we observed a close spatial alignment between these two effects, β-band desynchronization emerged later, and lasted substantially longer than the motor-evoked responses. A main exception was region 7a, which showed strong movement-related responses, but very little β-band desynchronization, highlighting the importance of simultaneous recording of activity from many different regions for full comprehension of neural dynamics underlying motor response.

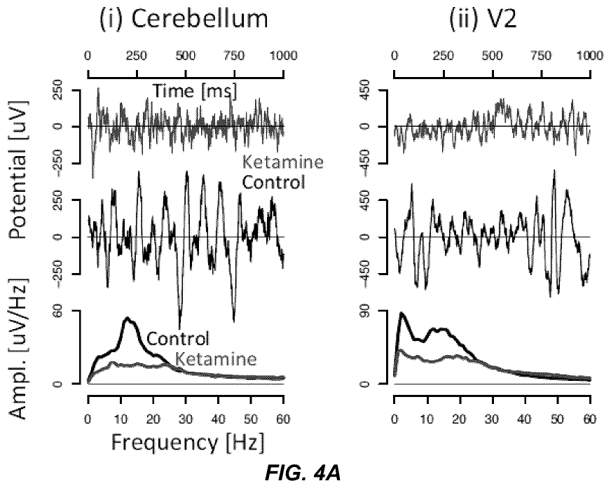

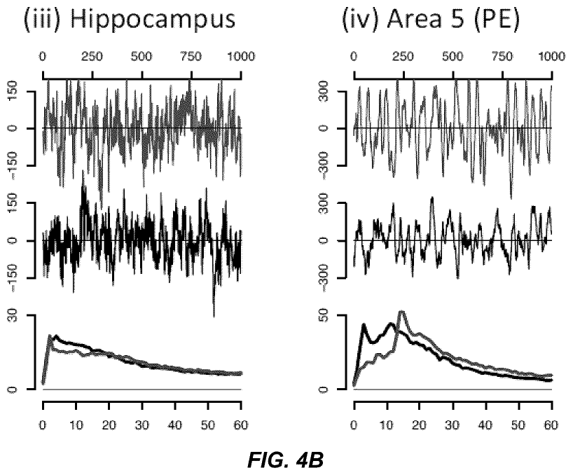

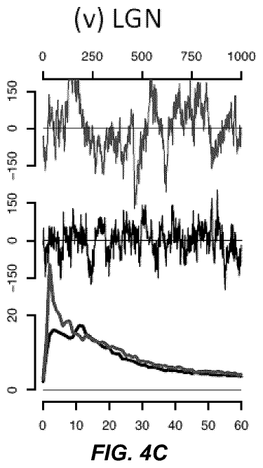

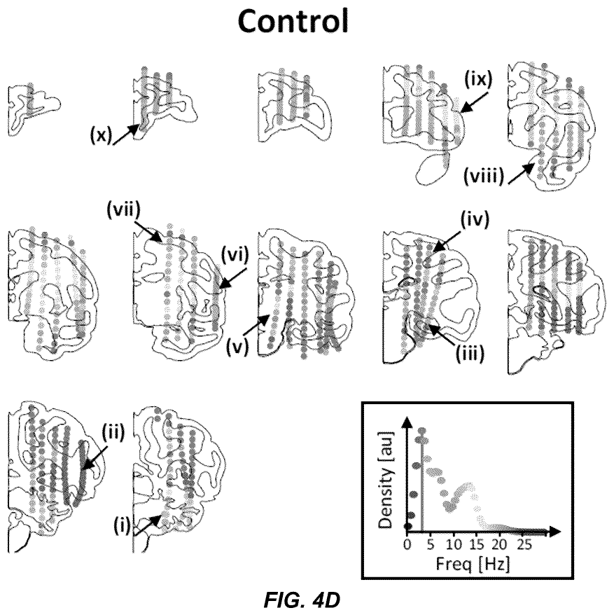

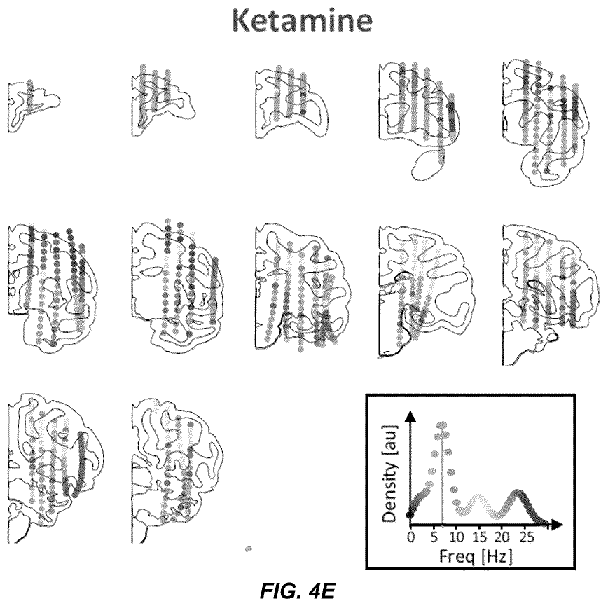

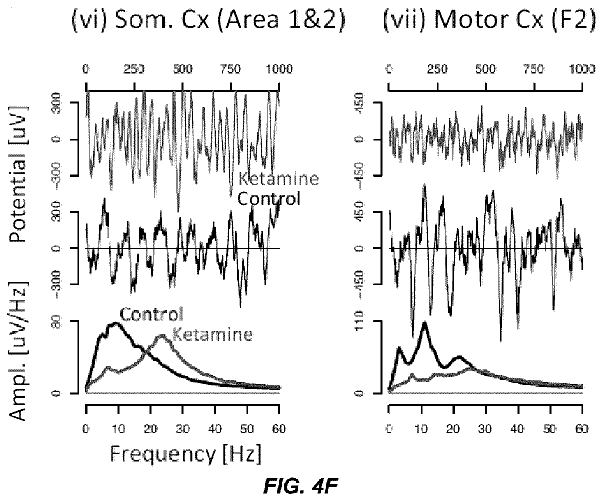

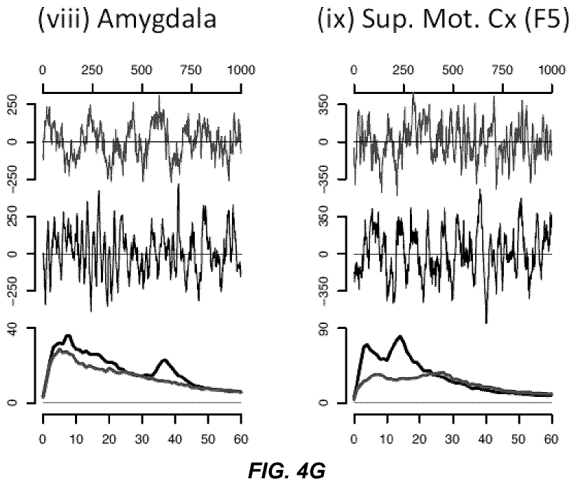

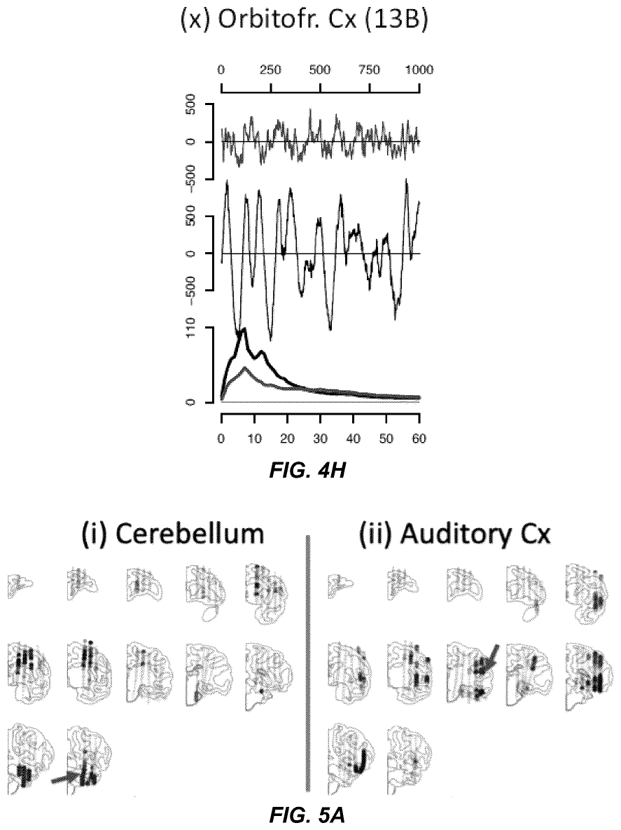

To highlight how MePhys can help understand intrinsic activity, we transitioned to resting-state recordings during 3- to 5-min. long blocks without presentation of stimuli or tasks, both at baseline and 5-10 min. after injection of subanesthetic doses of 1) ketamine and 2) midazolam. A frequency analysis during rest revealed spectra with a wide range of different shapes that varied systematically with electrode location (FIGS. 4A-4H, black lines). Many spectra were dominated by a single peak. In other cases, we observed two or three distinct peaks, whereas others followed a 1/f-type decay. For each channel, we defined the dominant frequency as the frequency with the highest power. During the control condition, the dominant frequency in occipital and parietal cortex was centered in the d band around 3 Hz (FIG. 4D). The dominant frequency in frontal cortex, basal ganglia, and cerebellum was centered in the low b band around 12 Hz. Finally, there were numerous contacts in prefrontal, orbitofrontal, and anterior temporal cortex with a dominant frequency in the high θ (or low α) range around 8 Hz. We did not identify any contacts with dominant frequencies above 25 Hz. However, contacts in and around ventral striatum, amygdala, and piriform cortex showed a narrow and distinct secondary peak in the c range centered on 40 Hz (FIG. 4Dviii). The key effect of ketamine was to reduce power, especially for frequencies below ˜20 Hz; ketamine also flattened previously identified spectral peaks (FIGS. 4A-4H, red lines). These effects were particularly striking in the cerebellum and motor cortex. Ketamine also eliminated the 40 Hz peak in and around the amygdala (FIG. 4Gviii). In contrast, the most striking effect of midazolam was the emergence and/or enhancement of ˜20 Hz peaks across large swaths of prefrontal cortex. Midazolam also tended to flatten out peaks of the spectra, but the effects were less pronounced than for ketamine. For example, the 40 Hz oscillations in the amygdala that were abolished by ketamine were not affected by midazolam.

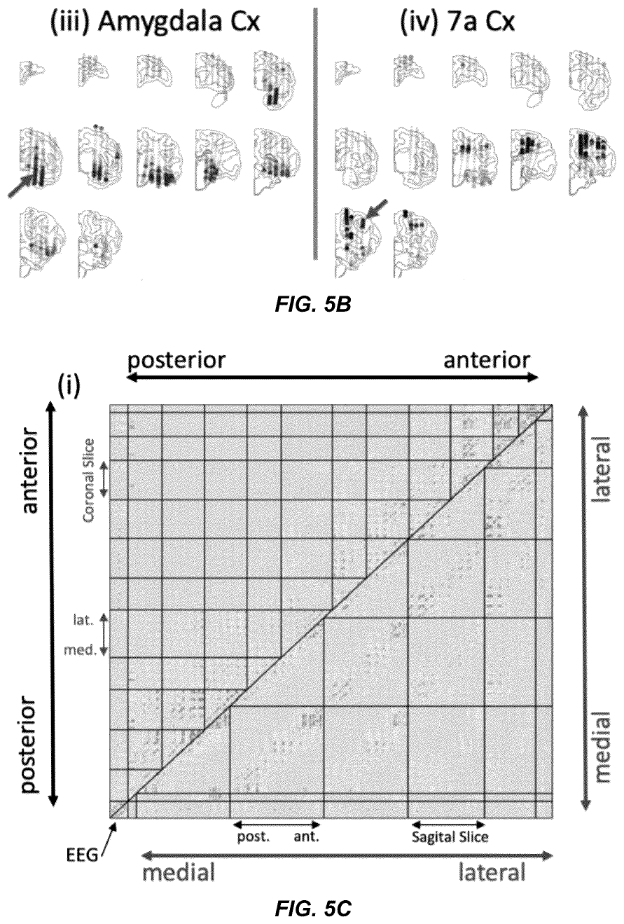

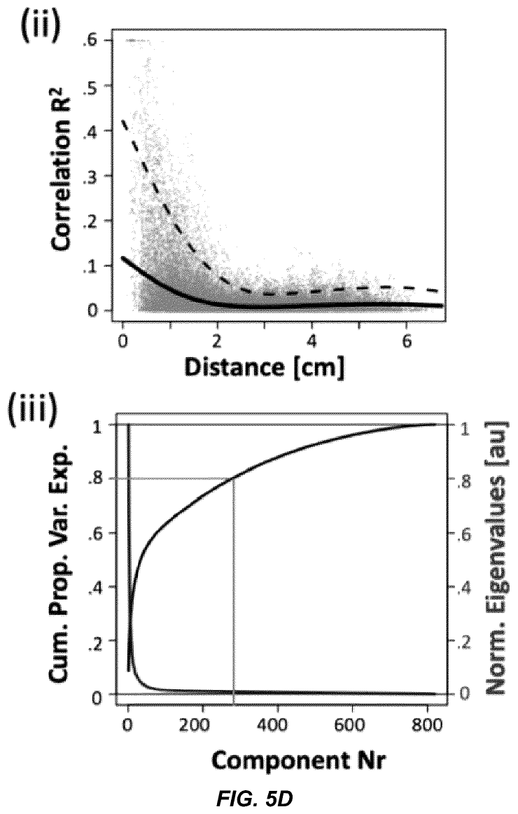

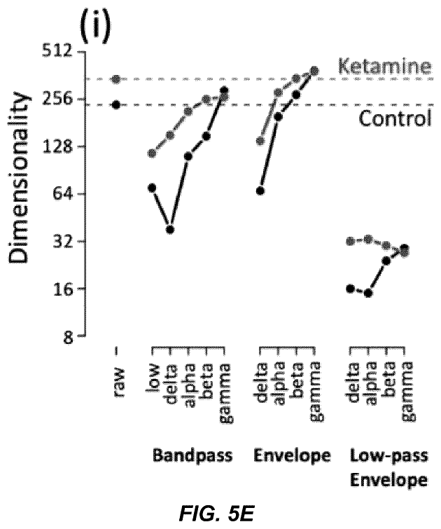

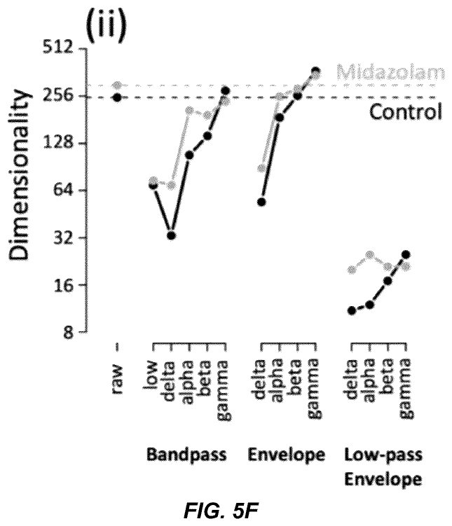

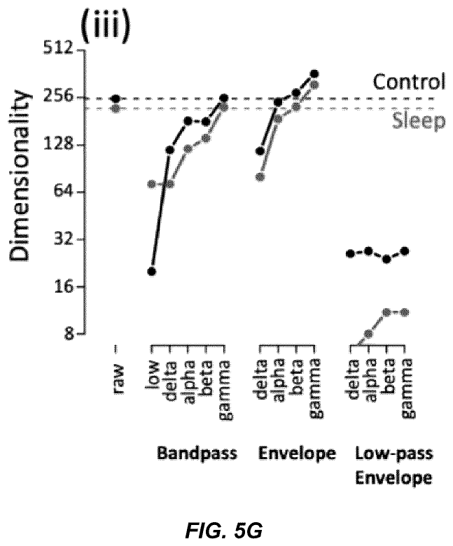

One of the key applications of MePhys is description of large-scale hemisphere-wide functional networks with high spectro-temporal resolution that is not available in classical functional magnetic resonance imaging (fMRI)-based functional connectivity analyses. FIGS. 5A-5B visualize four such functional connectivity maps using example seed channels in the cerebellum, auditory cortex, amygdala, and the temporo-parietal junction. As a next step, we computed the fullmatrix of temporal correlations for all >300,000 simultaneously recorded pairs of channels. Following the nomenclature in the fMRI field, we refer to this matrix as the (mesoscopic) functional connectome. FIG. 5C visualizes the functional connectome for the raw data in the control condition prior to injection of ketamine. The analysis identified a wide range of positive and negative correlations with meaningful spatial variation. The average value of the correlations (spearman correlation r2) decreased with distance, dropping close to zero at ˜2 cm (FIG. 5D, top panel). We defined the dimensionality of the connectome as the number of components needed to explain 80% of the variance (FIG. 5D, bottom panel).

Because brain function and communication has been suggested to occur in specific frequency bands, we filtered raw LFP data in five frequency bands: low (0.1-1 Hz), δ (2-4 Hz), θ/low α (5-10 Hz), low β/μ(10-20 Hz) and low γ (20-40 Hz). Frequencies below 0.1 Hz did not contain any meaningful signal because of the hardware filter of the amplifier. We also calculated the envelopes for four band-passed data sets (δ, α, β, γ) by low-pass filtering the full-wave rectified band-passed data. It has been suggested that power fluctuations in certain frequency bands may be a homolog of blood oxygen-level dependent (BOLD) fluctuations. Hence, we created two versions of the envelopes with either a high-or lowpass filter of 0.1 Hz. Using a seed-channel in cerebellum, as an example, we show that the functional connectivity maps indeed depend on the 14 different filter settings.

To better quantify the dimensionality of the data extracted by the different filter settings, we computed the functional connectomes for the different data sets. Dimensionality of the connectomes varied systematically as a function of carrier frequency, mode, and experimental condition (FIGS. 5E-5G). 1) With few exceptions, dimensionality increased with carrier frequency. 2) Dimensionality of the low-passed envelope is markedly lower than the bands and envelopes. 3) Ketamine increased dimensionality for almost all carrier frequencies and modes. A similar but smaller amplitude effect was observed for midazolam. In contrast to ketamine and midazolam, sleep decreased the dimensionality of the connectomes.

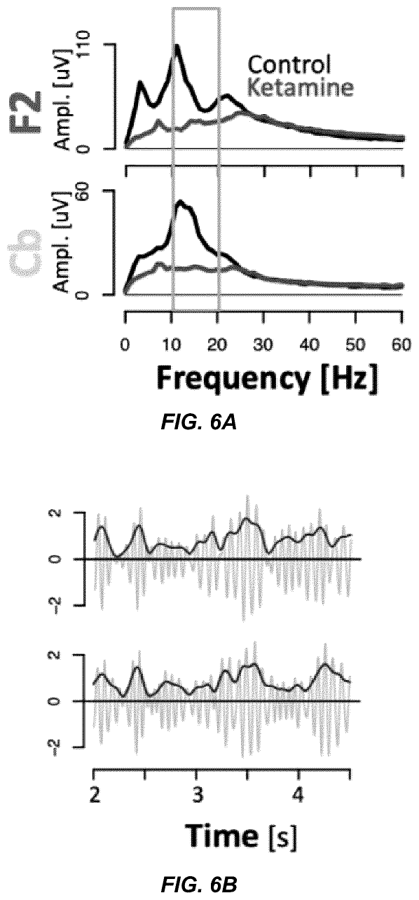

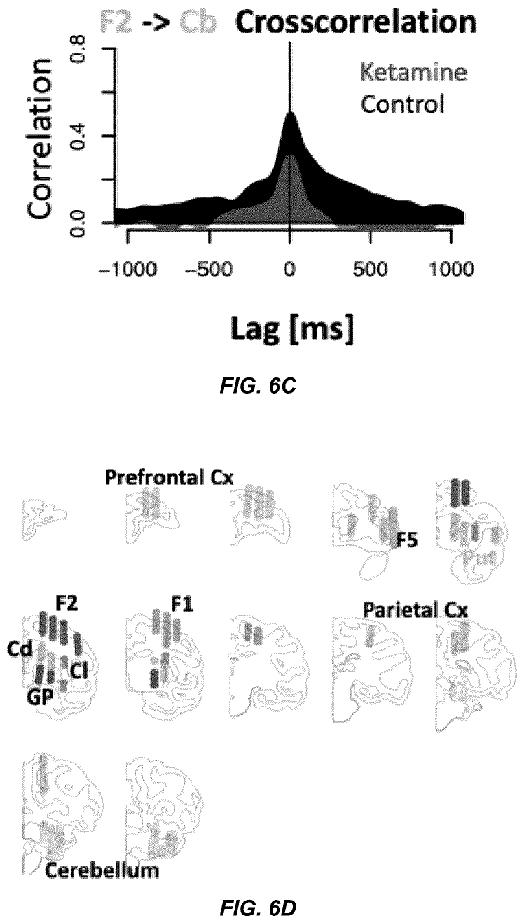

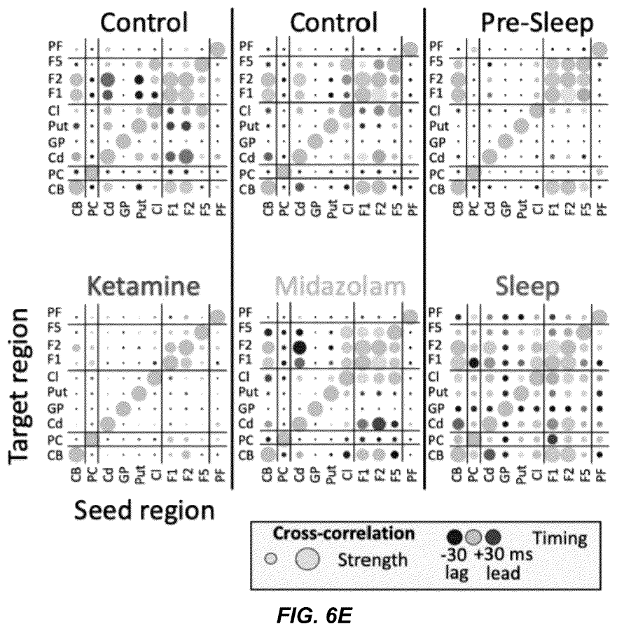

We furthermore leveraged the high temporal resolution of MePhys to quantify the temporal lag between different brain regions. As an example, we focused on β-band coupling in several cortical and subcortical brain regions associated with motor control. For example, our cross-correlation analyses show that β-band envelopes in motor cortex lead cerebellum by ˜2 ms (FIGS. 6A-6E). This tight temporal coupling suggests that the communication between the two structures is fast enough to play a role for online motor control. Such spectral and temporal specificity cannot be identified using methods that lack millisecond temporal resolution. The results further show that ketamine reduces β-band cross-correlation between the brain regions associated with motor control. No such decoupling was observed after injection of midazolam or during sleep (FIG. 6E).

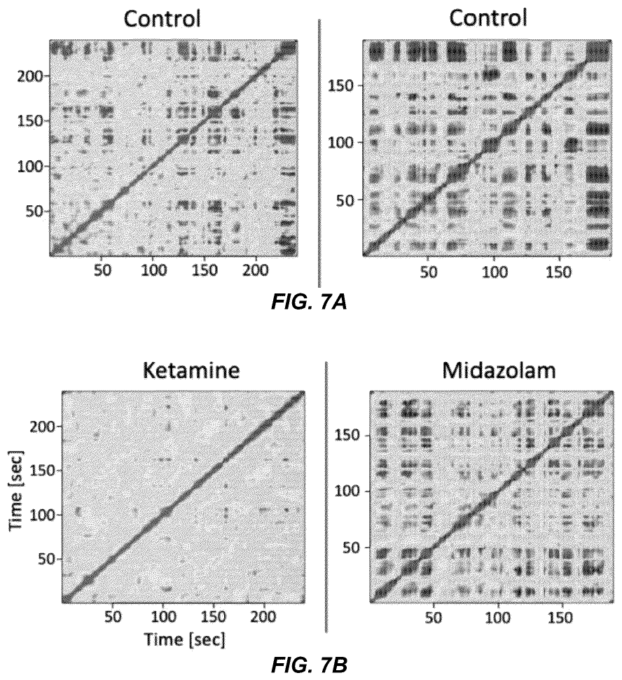

Another promising application for MePhys is the ability to study if and how the brain transitions through different intrinsic states over time. The low dimensionality of the low-passed envelopes may reflect a small number of intrinsic states mediated by slow-acting transmitter systems (neuropeptides), or slowly fluctuating physiological (hunger, thirst, etc.), emotional (fear, anxiety, etc.), or cognitive states (attention, arousal, etc.). We concatenated the four low-passed envelopes and calculated spatial correlations between all pairs of time-points. As the temporal distance between two sets of maps increases, their similarity tends to decrease. However, our results also show that between periods of change, the brain settles into relatively stable states for periods on the order of tens of seconds (FIG. 7A). The patches of strong positive correlations visible off of the main diagonal suggest that the same stable patterns tend to recur at different points in time. The block-like structure also suggests rapid transitions between different intrinsic states. Interestingly, this entire structure is almost completely abolished following the injection of ketamine (FIG. 7B, left panel). In contrast, the injection of midazolam had no effect on the emergence and recurrence of stable states (FIG. 7B, right panel).

The data presented earlier was collected within the first 3 months of having implanted all MePhys electrode shafts. By now, the electrodes have been implanted for at least 18 months, thus allowing us to provide a first glimpse at the longitudinal stability of the recordings. However, the scope of the present work precludes a conclusive analysis of longitudinal stability, which will be the topic of a separate manuscript.

As expected for chronically implanted electrodes, most single cells disappeared within ˜3 months of electrode implantation. In those initial 3 months, however, the single-cell recordings were of high signal-to-noise ratio and appeared to be extremely stable over the course of weeks. Analog multiunit activity can still be recorded reliably from most of the electrodes that picked up multiunit activity to begin with. Given the focus of MePhys on LFPs, we quantified the stability of the LFP data in more detail. Longitudinal stability was analyzed using two separate metrics: 1) amplitude spectra during rest and 2) amplitude of auditory-evoked potentials. In both cases, we compared data collected in August and September of 2023 to data collected in August and September of 2024.

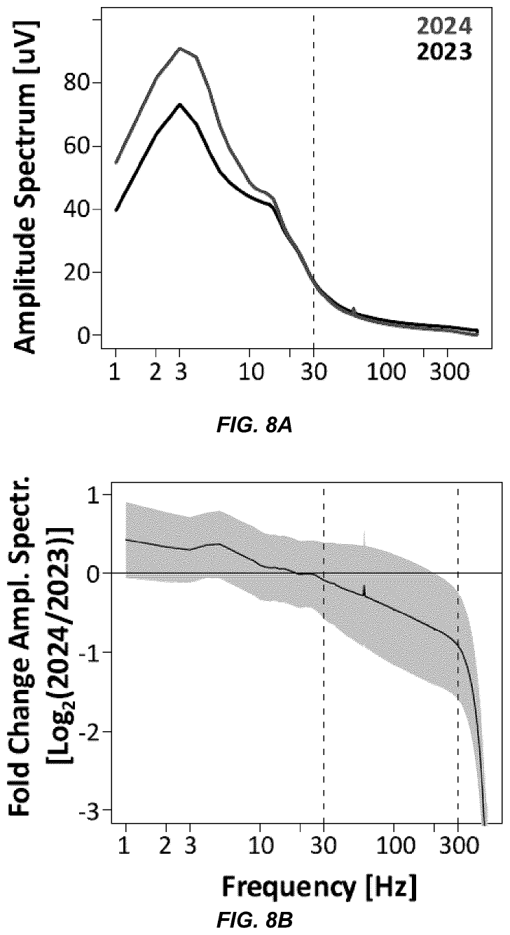

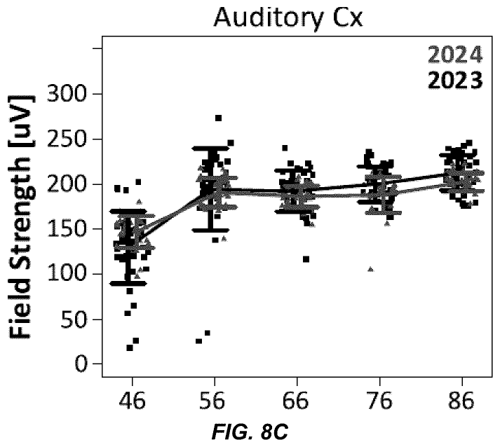

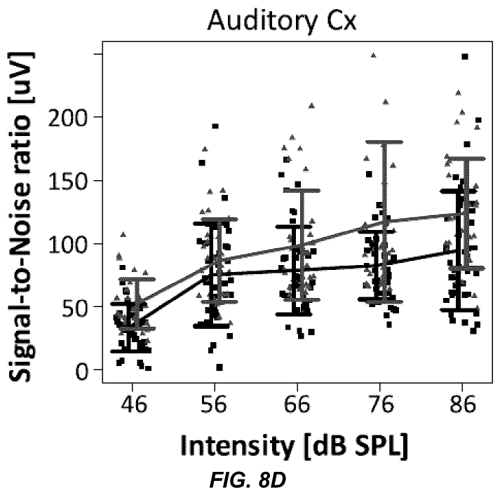

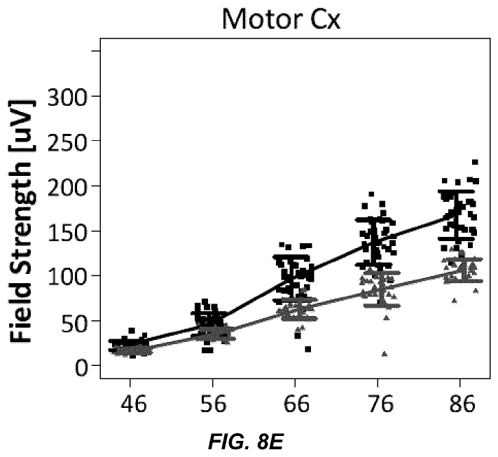

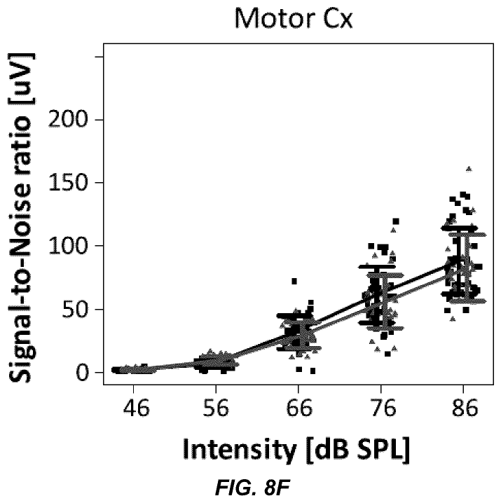

Our resting-state analysis showed that on average across all channels, power of classical LFP bands (below ˜100 Hz) remained comparable across the 1-yr time-period (FIGS. 8A-8B). Power below 30 Hz was somewhat higher in 2024, whereas power above 30 Hz was somewhat lower. However, the strongest and most pronounced reduction was observed for frequencies above 300 Hz. This is consistent with the notion of scar-tissue formation around the electrodes. The increase of power below 30 Hz may reflect motivational factors that are impossible to control over such a long timeframe. In line with this interpretation, we found strong and systematic modulations between data sets collected less than 3 h. apart at the beginning and end of each recording session, i.e., when the animal was either motivated to work or sated (data not shown).

The analysis of the auditory-evoked electric fields (AEFs) showed that neural response amplitude of channels in and around primary auditory cortex (A1) was largely unchanged over the 1-yr period (FIG. 8C). Interestingly, we found a very pronounced reduction of AEF amplitudes for channels in and around motor cortex (FIG. 8E). There are a couple of potential explanations. 1) It is possible that auditory responses in motor cortex are more dependent on attention and may, thus, have declined over time as the animal got more accustomed to the setting and the paradigm. 2) Because AEFs in motor cortex, unlike A1, showed a strong and almost linear increase with sound intensity, it is possible that the reduction of AEF amplitude in motor cortex was driven by an unintentional reduction of sound intensities, i.e., technical problems with sound delivery. 3) It is also possible that the effects were caused by a gradual degradation of the tissue-electrode interface. Given that the effect was only observed at channels in motor cortex, but not in auditory cortex, this explanation seems less likely. However, it is possible that some channels are more susceptible and that these channels just happened to be in motor cortex.

To better understand the effect on the quality of the recorded data we turned to signal-to-noise ratio, defined as the ratio of AEF power from 0 to 70 ms after sound onset and AEF power from 140 to 70 ms before sound onset. We found that signal-to-noise ratio remained almost constant in auditory cortex (FIG. 8D). Furthermore, in motor cortex, the difference of signal-to-noise ratio between 2023 and 2024 was much less pronounced than the reduction in AEF amplitude. Thus, the drop in signal amplitude was partially compensated by a drop in noise amplitude. It is unclear what caused the drop of noise amplitude. Based on the finding of increased spectral power below 30 Hz during rest, one could have expected an increase in noise amplitude on the baseline of the AEFs. However, since spectral amplitude was measured during rest, rather than during the auditory paradigm, these findings may not necessarily translate. A second potential explanation is based on the premise that part of the baseline noise may reflect the tail end of the auditory-evoked response to the previous sound. In that case, the reduction of noise might correspond to a reduction of the longer-latency aspects of the AEFs. Since long-latency responses tend to be associated with more cognitive aspects, this may reflect a gradual reduction of cognitive engagement with the same paradigm over the 1-yr period. However, it seems likely that much of the response to the previous sound would be averaged out, given that the intertrial interval was jittered.

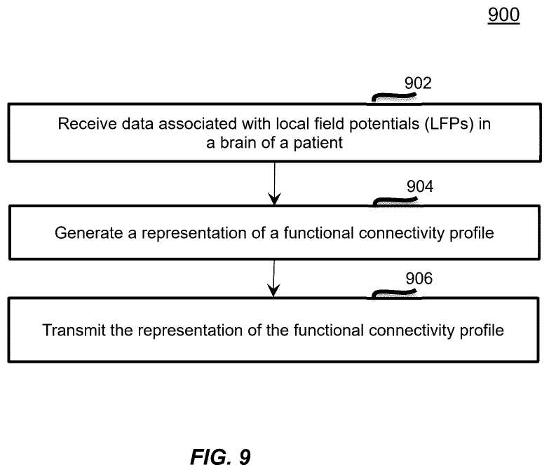

Referring now to FIG. 9, shown is a flow diagram of a process 900 for measuring LFPs in a brain of a patient using mesoscopic electrophysiological system 100, according to some non-limiting embodiments. The steps shown in FIG. 9 are for example purposes only. It will be appreciated that additional, fewer, different, and/or different order of steps may be used in non-limiting embodiments or aspects. In some non-limiting embodiments or aspects, a step may be automatically performed in response to performance and/or completion of a prior step. In some non-limiting embodiments or aspects, process 900 may be performed during a training process. In some non-limiting embodiments or aspects, one or more of the steps of process may be performed (e.g., completely, partially, and/or the like) by mesoscopic electrophysiological system 100 (e.g., at least one computing device of mesoscopic electrophysiological system 100). In some non-limiting embodiments or aspects, one or more of the steps of process 900 may be performed (e.g., completely, partially, and/or the like) by another system, another device, another group of systems, or another group of devices, separate from or including mesoscopic electrophysiological system 100.

As shown in FIG. 9, at step 902, process 900 may include receiving data associated with local field potentials (LFPs) in a brain of a patient. For example, a computing device of mesoscopic electrophysiological system 100 may receive data associated with LFPs in a brain of a patient based on measurements of LFPS by a plurality of electrode contacts on a plurality of electrode shafts implanted in the brain of the patient.

As shown in FIG. 9, at step 904, process 900 may include generating a representation of a functional connectivity profile. For example, a computing device of mesoscopic electrophysiological system 100 may generate the representation of the functional connectivity profile of the brain of the patient. In some non-limiting embodiments, a functional connectivity profile of the brain may include a map of statistical dependencies between different brain regions, showing how activity in the different brain regions is synchronized over time. The functional connectivity profile may show how the different brain regions interact and/or communicate, reflecting patterns of activity that underlie cognitive processes, emotions, and/or behavior.

As shown in FIG. 9, at step 906, process 900 may include transmitting the representation of the functional connectivity profile. For example, a computing device of mesoscopic electrophysiological system 100 may transmit the representation of the functional connectivity profile as a graph to another computing device (e.g., of mesoscopic electrophysiological system 100 or another computing device of a different system).

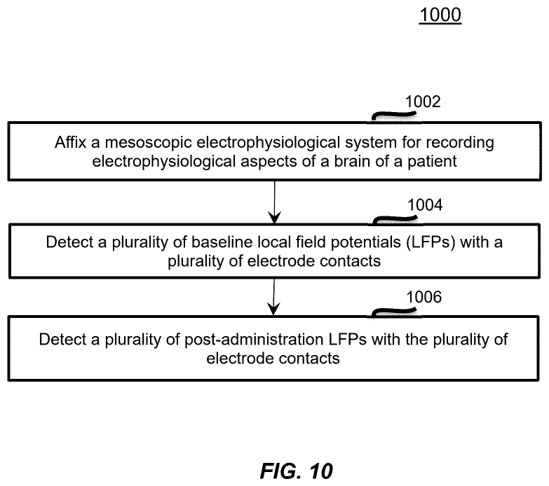

Referring now to FIG. 10, shown is a flow diagram of a method for monitoring a plurality of LFPs within a brain of a patient, according to some non-limiting embodiments. The steps shown in FIG. 10 are for example purposes only. It will be appreciated that additional, fewer, different, and/or different order of steps may be used in non-limiting embodiments or aspects. In some non-limiting embodiments or aspects, a step may be automatically performed in response to performance and/or completion of a prior step. In some non-limiting embodiments or aspects, process 1000 may be performed during a training process. In some non-limiting embodiments or aspects, one or more of the steps of process may be performed (e.g., completely, partially, and/or the like) by mesoscopic electrophysiological system 100 (e.g., at least one computing device of mesoscopic electrophysiological system 100). In some non-limiting embodiments or aspects, one or more of the steps of process 900 may be performed (e.g., completely, partially, and/or the like) by another system, another device, another group of systems, or another group of devices, separate from or including mesoscopic electrophysiological system 100.

As shown in FIG. 10, at step 1002, process 1000 may include affixing a mesoscopic electrophysiological system for recording electrophysiological aspects of a brain of a patient. For example, one or more components of structural system 102 of mesoscopic electrophysiological system 100 may be affixed to a patient.

As shown in FIG. 10, at step 1004, process 1000 may include detecting a plurality of baseline local field potentials (LFPs) with a plurality of electrode contacts. For example, a computing device of mesoscopic electrophysiological system 100 may detect a plurality of baseline LFPs with a plurality of electrode contacts.

As shown in FIG. 10, at step 1006, process 1000 may include detecting a plurality of post-administration LFPs with the plurality of electrode contacts. For example, a computing device of mesoscopic electrophysiological system 100 may detect a plurality of post-administration LFPs with the plurality of electrode contacts based on (e.g., after) administering a compound of interest to the patient.

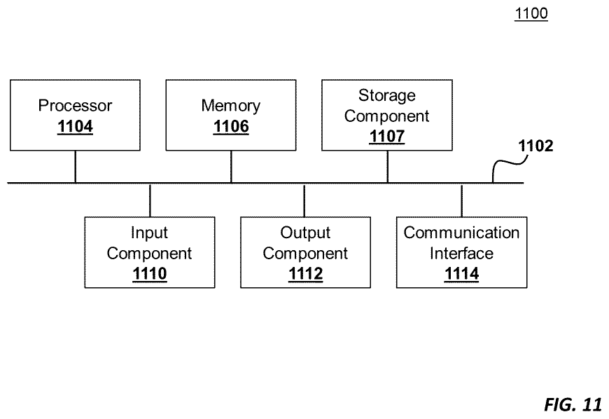

Referring now to FIG. 11, shown is a diagram of example components of device 1100, which may be a component of a mesoscopic electrophysiological system, according to non-limiting embodiments or aspects. Device 1100 may correspond to at least one computing device of mesoscopic electrophysiological system 100 (e.g., one or more components of mesoscopic electrophysiological system 100) as described herein. In some non-limiting embodiments or aspects, such computing devices may include at least one device 1100 and/or at least one component of device 1100. The number and arrangement of components shown in FIG. 11 are provided as an example. In some non-limiting embodiments or aspects, device 1100 may include additional components, fewer components, different components, or differently arranged components than those shown in FIG. 11. Additionally or alternatively, a set of components (e.g., one or more components) of device 1100 may perform one or more functions described as being performed by another set of components of device 1100.

As shown in FIG. 11, device 1100 may include bus 1102, processor 1104, memory 1106, storage component 1107, input component 1110, output component 1112, and communication interface 1114. Bus 1102 may include a component that permits communication among the components of device 1100. In some non-limiting embodiments or aspects, processor 1104 may be implemented in hardware, firmware, or a combination of hardware and software. For example, processor 1104 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 1106 may include random access memory (RAM), read only memory (ROM), and/or another type of dynamic or static storage device (e.g., flash memory, magnetic memory, optical memory, etc.) that stores information and/or instructions for use by processor 1104.

With continued reference to FIG. 11, storage component 1107 may store information and/or software related to the operation and use of device 1100. For example, storage component 1107 may include a hard disk (e.g., a magnetic disk, an optical disk, a magneto-optic disk, a solid-state disk, etc.) and/or another type of computer-readable medium. Input component 1110 may include a component that permits device 1100 to receive information, such as via user input (e.g., a touch screen display, a keyboard, a keypad, a mouse, a button, a switch, a microphone, etc.). Additionally or alternatively, input component 1110 may include a sensor for sensing information (e.g., a global positioning system (GPS) component, an accelerometer, a gyroscope, an actuator, etc.). Output component 1112 may include a component that provides output information from device 1100 (e.g., a display, a speaker, one or more light-emitting diodes (LEDs), etc.). Communication interface 1114 may include a transceiver-like component (e.g., a transceiver, a separate receiver and transmitter, etc.) that enables device 1100 to communicate with other devices, such as via a wired connection, a wireless connection, or a combination of wired and wireless connections. Communication interface 1114 may permit device 1100 to receive information from another device and/or provide information to another device. For example, communication interface 1114 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 1100 may perform one or more processes described herein. Device 1100 may perform these processes based on processor 1104 executing software instructions stored by a computer-readable medium, such as memory 1106 and/or storage component 1107. A computer-readable medium may include any non-transitory memory device. A memory device includes memory space located inside of a single physical storage device or memory space spread across multiple physical storage devices. Software instructions may be read into memory 1106 and/or storage component 1107 from another computer-readable medium or from another device via communication interface 1114. When executed, software instructions stored in memory 1106 and/or storage component 1107 may cause processor 1104 to perform one or more processes described herein. Additionally or alternatively, hardwired circuitry may be used in place of or in combination with software instructions to perform one or more processes described herein. Thus, embodiments described herein are not limited to any specific combination of hardware circuitry and software. The term “configured to,” as used herein, may refer to an arrangement of software, device(s), and/or hardware for performing and/or enabling one or more functions (e.g., actions, processes, steps of a process, and/or the like). For example, “a processor configured to” may refer to a processor that executes software instructions (e.g., program code) that cause the processor to perform one or more functions.

Although embodiments have been described in detail for the purpose of illustration, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

What is claimed is

1. A mesoscopic electrophysiological system for recording electrophysiological aspects of a brain of a patient, comprising:
a structural system sized and configured to be attached to a head of a patient and to hold a plurality of intracranial electrode shafts in predetermined positions around the head of the patient;
a plurality of mesoscopic intracranial electrode shafts that comprises a first plurality of electrode contacts configured to measure local field potentials (LFPs) at a plurality of locations in a brain of a patient, wherein each electrode shaft of the plurality of intracranial electrode shafts comprises a second plurality of electrode contacts; and
a plurality of guide pins sized and configured to receive the plurality of mesoscopic intracranial electrode shafts positioned therein;
wherein the first plurality of electrode contacts forms a three-dimensional grid within the head of the patient.

2. The mesoscopic electrophysiological system of claim 1, wherein the second plurality of contacts is configured to provide electrical micro-stimulation to the plurality of locations in the brain of the patient.

3. The mesoscopic electrophysiological system of claim 1, wherein each guide pin of the plurality of guide pins comprises an interior channel, and wherein a sterile grease is provided in the interior channel of one or more guide pins of the plurality of guide pins.

4. The mesoscopic electrophysiological system of claim 3, wherein each guide pin of the plurality of guide pins comprises a vent connected to the interior channel, and wherein the vent provides a path for the sterile grease to travel when a mesoscopic intracranial electrode shaft is positioned within the interior channel of a guide pin.

5. The mesoscopic electrophysiological system of claim 1, wherein the second plurality of electrode contacts have variable inter-electrode spacing in range between 250 μm and 3.5 mm.

6. The mesoscopic electrophysiological system of claim 1, further comprising:
at least one processor configured to:
receive data associated with the LFPs in the brain of the patient from a subset of the first plurality of electrode contacts;
generate a representation of a functional connectivity profile for the patient based on the data associated with the LFPs in the brain of the patient; and
transmit the representation of the functional connectivity profile for the patient to a communications device.

7. The mesoscopic electrophysiological system of claim 1, wherein the first plurality of electrode contacts is configured to provide at least 1,000 channels for measuring the LFPs at the plurality of locations in the brain of the patient.

8. The mesoscopic electrophysiological system of claim 1, wherein the first plurality of electrode contacts is configured to measure the LFPs at the plurality of locations in the brain of the patient at a sampling rate in a range between 20 kHz and 30 kHz.

9. The mesoscopic electrophysiological system of claim 1, wherein the structural system comprises:
a crown component comprising a top section and an annular section, wherein the crown component is configured to be anchored to a skull of the patient via fasteners inserted through the annular section;
a semi-spherical grid component comprising a first structure having a first plurality of apertures, wherein the structure corresponds to a shape of a skull of the patient, and wherein the semi-spherical grid component is positioned within the crown component;
a wall component comprising a base section and a top section, wherein the base section is configured to connect to the top section of the crown component; and
a grid component comprising a second structure having a second plurality of apertures, wherein the grid component is configured to fit within the wall component and is configured to receive the plurality of guide pins within the second plurality of apertures, and wherein the second plurality of apertures align with at least a portion of the first plurality of apertures to allow for the plurality of intracranial electrode shafts to be inserted into the brain of the patient.

10. The mesoscopic electrophysiological system of claim 9, wherein the second plurality of apertures of the grid component are spaced apart in an antero-posterior direction and medio-later direction by a dimension in a range between 4 mm and 5 mm.

11. The mesoscopic electrophysiological system of claim 9, wherein one or more apertures of the second plurality of apertures has an inner diameter equal to 1.5 mm.

12. The mesoscopic electrophysiological system of claim 9, wherein the structural system comprises:
a cap component comprising a cap section and a cap top section, wherein the cap section comprises a wall having a peripheral aperture, wherein the wall defines a main aperture, wherein the cap section is configured to connect to the top section of the wall component, wherein the cap top section is configured to cover the main aperture of the wall, and wherein the peripheral aperture is sized and configured to allow for cables that connect to the intracranial electrode shafts to pass therethrough.

13. The mesoscopic electrophysiological system of claim 5, wherein the semi-spherical grid component comprises a plurality of electroencephalogram (EEG) electrodes that are positioned to contact a surface of the head of the patient when the structural system is placed on the head of the patient.

14. A mesoscopic electrophysiological system for recording electrophysiological aspects of a brain of a patient, comprising:
a structural system sized and configured to be attached to a head of a patient and to hold a plurality of intracranial electrode shafts in predetermined positions around the head of the patient;
a plurality of mesoscopic intracranial electrode shafts that comprises a plurality of electrode contacts configured to measure local field potentials (LFPs) at a plurality of locations in a brain of a patient; and
a plurality of guide pins sized and configured to receive the plurality of mesoscopic intracranial electrode shafts positioned therein;
wherein the plurality of electrode contacts forms a three-dimensional grid within the head of the patient.

15. The mesoscopic electrophysiological system of claim 14, wherein each guide pin of the plurality of guide pins comprises an interior channel, and wherein a sterile grease is provided in the interior channel of one or more guide pins of the plurality of guide pins.

16. The mesoscopic electrophysiological system of claim 15, wherein each guide pin of the plurality of guide pins comprises a vent connected to the interior channel, and wherein the vent provides a path for the sterile grease to travel when a mesoscopic intracranial electrode shaft is positioned within the interior channel of a guide pin.

17. The mesoscopic electrophysiological system of claim 14, wherein the second plurality of electrode contacts have variable inter-electrode spacing in range between 250 μm and 3.5 mm.

18. The mesoscopic electrophysiological system of claim 14, wherein the first plurality of electrode contacts is configured to provide at least 1,000 channels for measuring the LFPs at the plurality of locations in the brain of the patient, and wherein the plurality of electrode contacts is configured to measure the LFPs at the plurality of locations in the brain of the patient at a sampling rate in a range between 20 kHz and 30 kHz.

19. A method of monitoring a plurality of local field potentials (LFPs) within a patient's brain, comprising:
affixing, to the patient, a mesoscopic electrophysiological system for recording electrophysiological aspects of a brain of a patient, the system comprising:
a structural system sized and configured to be attached to a head of a patient and to hold a plurality of intracranial electrode shafts in predetermined positions around the head of the patient;
a plurality of mesoscopic intracranial electrode shafts that comprises a plurality of electrode contacts configured to measure the LFPs at a plurality of locations in a brain of a patient, the plurality of electrode contacts forming a three-dimensional grid within the head of the patient;
a plurality of guide pins sized and configured to receive the plurality of mesoscopic intracranial electrode shafts positioned therein;
at least one processor configured to:
receive data associated with the LFPs in the brain of the patient from a subset of the first plurality of electrode contacts;
generate a representation of a functional connectivity profile for the patient based on the data associated with the LFPs in the brain of the patient; and
transmit the representation of the functional connectivity profile for the patient to a communications device; and
detecting, with the plurality of electrode contacts, a plurality of baseline LFPs.

20. The method of claim 19, further comprising, after detecting the plurality of baseline LFPs:
administering to the patient a compound of interest and detecting, with the first plurality of electrode contacts, a plurality of post-administration LFPs.
